(19) United States
(12) Patent Application Publication
(10) Pub. No.: US 2006/0167564 A1
(43) Pub. Date: Jul. 27, 2006

(54) Limb and digit movement system

(76) Inventors: J. Christopher Flaherty, Topsfield, MA (US); R. Maxwell Flaherty, Topsfield, MA (US); Mijail D. Serruya, Providence, RI (US); Burke T. Barrett, Franklin, MA (US); Gerhard M. Friehs, East Greenwich, RI (US)

Correspondence Address: Leslie I. Bookoff;FINNEGAN, HENDERSON, FARABOW,, GARRETT & DUNNER, L.L.P., 901 New York Avenue, N.W., Washington, DC, 20001-4413, US

(21) Appl. No.: 11/319,703

(22) Filed: Dec. 29, 2005

Related U.S. Application Data
(60) Provisional application No. 60/642,810, filed on Jan. 10, 2005.

(51) Int. Cl. A61F 2/66

(52) U.S. Cl. 623/57

(57) ABSTRACT

Systems, methods and devices for restoring or enhancing one or more motor functions of a patient are disclosed. The system comprises a biological interface apparatus and a joint movement device such as an exoskeleton device or FES device. The biological interface apparatus includes a sensor that detects the multicellular signals and a processing unit for producing a control signal based on the multicellular signals. Data from the joint movement device is transmitted to the processing unit for determining a value of a configuration parameter of the system. Also disclosed is a joint movement device including a flexible structure for applying force to one or more patient joints, and controlled cables that produce the forces required.

This application claims the benefit of priority under 35 U.S.C. § 119(e) of U.S. Provisional Application No. 60/642,810, filed Jan. 10, 2005. This application relates to commonly assigned U.S. Application Ser. No. ______ of J. Christopher Flaherty et al., entitled “JOINT MOVEMENT APPARATUS” and filed on the same date as the present application. The complete subject matter of the above-referenced applications is incorporated by reference herein.

### FIELD OF THE INVENTION

The present invention relates generally to medical devices, systems and methods for restoring or enhancing one or more motor functions of a patient, and more particularly to systems, methods and devices for extracting signals directly from one or more cells of a patient, such as nerve cells of the human brain, to create a control signal.

### DESCRIPTION OF RELATED ART

Biological interface devices, for example neural interface devices, are currently under development for numerous patient applications including restoration of lost function due to traumatic injury or neurological disease. Sensors, such as electrode arrays, implanted in the higher brain regions that control voluntary movement, can be activated voluntarily to generate electrical signals that can be processed by a biological interface device to create a thought invoked control signal. Such control signals can be used to control numerous devices including computers and communication devices, external prostheses, such as an artificial arm or functional electrical stimulation of paralyzed muscles, as well as robots and other remote control devices. Patient's afflicted with amyotrophic lateral sclerosis (Lou Gehrig's Disease), particularly those in advanced stages of the disease, would also be appropriate for receiving a neural interface device, even if just to improve communication to the external world, including Internet access, and thus improve their quality of life.

Early attempts to utilize signals directly from neurons to control an external prosthesis encountered a number of technical difficulties. The ability to identify and obtain stable electrical signals of adequate amplitude was a major issue. Another problem that has been encountered is caused by the changes that occur to the neural signals that occur over time, resulting in a degradation of system performance. Neural interface apparatus that utilize other neural information, such as electrocorticogram (ECOG) signals, local field potentials (LFPs) and electroencephalogram (EEG) signals have similar issues to those associated with individual neuron signals. Since all of these signals result from the activation of large groups of neurons, the specificity and resolution of the control signal that can be obtained is limited. However, if these lower resolution signals could be properly identified and the system adapt to their changes over time, simple control signals could be generated to control rudimentary devices or work in conjunction with the higher power control signals processed directly from individual neurons.

Commercialization of these neural interfaces has been extremely limited, with the majority of advances made by universities in a preclinical research setting. As the technologies advance and mature, the natural progression will be to more sophisticated human applications, such as those types of devices regulated by various governmental regulatory agencies including the Food and Drug Administration in the United States.

As sophisticated biological interface apparatus are approved by the FDA and become commercially available, these systems will be used with other assistive devices, such as powered exoskeletons, to restore a function of paraplegic, quadriplegic and other motor impaired patients. In order to provide safe and reliable movement assist systems, information transfer and other cooperation between components will be required to create a robust and predictable system. These systems must be self-monitoring and handle malfunctions in a manner to prevent injury. Simplified use, as well as convenience and flexibility to the patient, their caregivers and family members will also be a requirement. There is therefore a need for an improved movement assist system and biological interface apparatus to adequately serve these patient populations.

### SUMMARY OF THE INVENTION

According to a first aspect of the invention, a biological interface apparatus for controlling a joint movement device is disclosed. The biological interface apparatus collects multicellular signals emanating from one or more living cells of a patient and transmits processed signals to the joint movement device. The biological interface apparatus includes a sensor for detecting multicellular signals, the sensor comprising a plurality of electrodes. The electrodes are designed to detect the multicellular signals. A processing unit is designed to receive the multicellular signals from the sensor and process the multicellular signals to produce the processed signals transmitted to the joint movement device. The joint movement device applies a force to one or more joints, such as a patient joint or a joint of a prosthetic device. Joint movement device data is transmitted to the processing unit and used to determine a value for a configuration parameter used to produce the processed signals.

The joint movement device is selected from the group consisting of a exoskeleton device, an FES device and a prosthetic limb. The joint movement device may be attached to the patient or implanted in the patient. The joint movement device includes a force generator, such as a motor or hydraulic or pneumatic pump. Numerous joints are applicable to the joint movement device of the present invention, such as a shoulder, elbow, wrist, finger joint, knee, ankle, a toe joint, metacarpophalangeal joint, interphalangeal joint, and temporomandibular joint. The joint movement device data can be received from one or more components of the apparatus, such as the joint movement device itself. The data may be analyzed or processed, and may be compared to a threshold such as an adjustable threshold. The data can be available prior to use of the joint movement device such as a time constant of the device, or require the use of the device such as a parameter that is specific to the patient and generated during a system configuration or physical therapy session. The data may be entered by an operator, such as a remote operator utilizing the Internet, or obtained and transmitted automatically by the system. In another preferred embodiment, the joint movement device includes one or more sensors that provide data relative to the joint movement device or other data.

According to a second aspect of the invention, a biological interface apparatus for controlling a joint movement device is disclosed. The biological interface apparatus collects multicellular signals emanating from one or more living cells of a patient and transmits processed signals to the joint movement device. The biological interface apparatus includes a sensor for detecting multicellular signals, the sensor comprising a plurality of electrodes. The electrodes are designed to detect the multicellular signals. A processing unit is designed to receive the multicellular signals from the sensor and process the multicellular signals to produce the processed signals transmitted to the joint movement device. The joint movement device applies a force to one or more joints, such as a patient joint or a joint of a prosthetic device. The joint movement device transmits joint movement device data to the processing unit.

According to a third aspect of the invention, a joint movement device for applying a force to a patient's joint is disclosed. The joint movement device includes a force translating structure that is in contact with a portion of the patient. A force producing assembly is operably attached to a proximal end of one or more control cables. The distal end of the control cables is fixedly attached to the force translating structure such that the force produced by the force producing assembly causes a resultant force to be applied to the patient's joint. In an alternative embodiment, the joint movement device further includes a torque generating assembly that applies a torsional force to an additional joint of the patient. In a preferred embodiment, the joint movement device has a glove configuration and is used to control the patient's wrist and fingers. The torque generating assembly preferably applies a controllable torque to the patient's elbow. In another preferred embodiment, a system includes the joint movement device and the biological interface apparatus of the present invention, wherein the processed signals of the biological interface are used to control the joint movement device.

According to a fourth aspect of the invention, a joint movement device for applying a force to a patient's joint is disclosed. The joint movement device includes an implanted piston assembly that comprises a piston, a housing that slidingly receives the piston, and a linear actuator for controllably advancing and retracting the piston. The piston assembly is fixedly attached to a first bone of the patient and a distal end of the piston is fixedly attached to a second bone of the patient. Advancing and retracting the piston applies force to a joint of the patient. In a preferred embodiment, a system includes the joint movement device and the biological interface apparatus of the present invention, wherein the processed signals of the biological interface are used to control the joint movement device.

Both the foregoing general description and the following detailed description are exemplary and are intended to provide further explanation of the embodiments of the invention as claimed.

### BRIEF DESCRIPTION OF THE DRAWINGS

The accompanying drawings, which are incorporated in and constitute a part of this specification, illustrate various embodiments of the present invention, and, together with the description, serve to explain the principles of the invention. In the drawings:

FIG. 1 illustrates a schematic view of an exemplary embodiment of a biological interface apparatus including a joint movement device, consistent with the present invention;

FIG. 2 illustrates a patient performing physical therapy after having been enabled by a movement assist system, consistent with the present invention;

FIG. 3 illustrates an exemplary embodiment of a wrist and finger joint movement device, consistent with the present invention;

FIG. 4 illustrates an exemplary embodiment of a portion of the biological interface apparatus consistent with the present invention wherein sensor electrodes are implanted in the brain of a patient and a portion of a processing unit is implanted on the skull of the patient;

FIG. 5 illustrates another exemplary embodiment of a biological interface apparatus consistent with the present invention wherein an operator configures the apparatus at the patient site;

FIG. 6 illustrates an exemplary embodiment of an elbow, wrist and finger joint movement device, consistent with the present invention;

FIG. 7 illustrates a schematic view of a biological interface apparatus including two sensors which produce a control signal for a joint movement device, consistent with the present invention;

FIG. 8 illustrates a physical therapist and a patient performing physical therapy after the patient has been enabled by a movement assist system, consistent with the present invention; and

FIG. 9 illustrates an implanted joint movement device including a piston assembly connected to two bones of the patient, consistent with the present invention.

### DESCRIPTION OF THE EMBODIMENTS

To facilitate an understanding of the invention, a number of terms are defined immediately herebelow.

Definitions

As used herein, the term “biological interface apparatus” refers to a neural interface apparatus or any apparatus that interfaces with living cells that produce electrical activity or cells that produce other types of detectable signals.

As used herein, the term “cellular signals” refers to subcellular signals, intracellular signals, extracellular signals, single cell signals and signals emanating from one or more cells. “Subcellular signals” refers to a signal derived from a part of a cell; a signal derived from one particular physical location along or within a cell; a signal from a cell extension, such as a dendrite, dendrite branch, dendrite tree, axon, axon tree, axon branch, pseudopod or growth cone; or signals from organelles, such as golgi apparatus or endoplasmic reticulum. “Intracellular signals” refers to a signal that is generated within a cell or by the entire cell that is confined to the inside of the cell up to and including the membrane. “Extracellular signals” refers to signals generated by one or more cells that occur outside of the cell(s). “Cellular signals” include but are not limited to signals or combinations of signals that emanate from any living cell. Specific examples of “cellular signals” include but are not limited to: neural signals; cardiac signals including cardiac action potentials; electromyogram (EMG) signals; glial cell signals; stomach cell signals; kidney cell signals; liver cell signals; pancreas cell signals; osteocyte cell signals; sensory organ cell signals such as signals emanating from the eye or inner ear; and tooth cell signals. “Neural signals” refers to neuron action potentials or spikes; local field potential (LFP) signals; electroencephalogram (EEG) signals; electrocorticogram signals (ECoG); and signals that are between single neuron spikes and EEG signals.

As used herein, “multicellular signals” refers to signals emanating from two or more cells, or multiple signals emanating from a single cell.

As used herein, “patient” refers to any animal, such as a mammal and preferably a human. Specific examples of “patients” include but are not limited to: individuals requiring medical assistance; healthy individuals; individuals with limited function; and in particular, individuals with lost motor or other function due to traumatic injury or neurological disease.

As used herein, “configuration” refers to any alteration, improvement, repair, calibration or other system-modifying event whether manual in nature or partially or fully automated.

As used herein, “configuration parameter” refers to a variable, or a value of a variable, of a component, device, apparatus and/or system. A configuration parameter has a value that can be: set or modified; used to perform a function; used in a mathematical or other algorithm; used as a threshold to perform a comparison; and combinations of these. A configuration parameter's value determines the characteristics or behavior of something. System configuration parameters are variables of the system of the present invention, such as those used to by the processing unit to produce processed signals. Other, numerous subsets of configuration parameters are applicable, these subsets including but not limited to: calibration parameters such as a calibration frequency parameter; controlled device parameters such as a time constant parameter; processing unit parameters such as a cell selection criteria parameter; patient parameters such as a patient physiologic parameter such as heart rate; multicellular signal sensor parameters; other sensor parameters; system environment parameters; mathematical algorithm parameters; a safety parameter; and other parameters. Certain parameters may be controlled by the patient's clinician, such as a password-controlled parameter securely controlled by an integral permission routine of the system. Certain parameters may represent a “threshold” such as a success threshold value used in a comparison to determine if the outcome of an event was successful. In numerous steps of a system configuration or other function, a minimum performance or other measure may be maintained by comparing a detected signal, or the output of an analysis of one or more signals, to a success threshold value.

As used herein, “discrete component” refers to a component of a system such as those defined by a housing or other enclosed or partially enclosed structure, or those defined as being detached or detachable from another discrete component. Each discrete component can transmit data to a separate component through the use of a physical cable, including one or more of electrically conductive wires or optical fibers, or transmission of data can be accomplished wirelessly. Wireless communication can be accomplished with a transceiver that may transmit and receive data such as through the use of “Bluetooth” technology or according to any other type of wireless communication means, method, protocol or standard, including, for example, code division multiple access (CDMA), wireless application protocol (WAP), Infrared or other optical telemetry, radio frequency or other electromagnetic telemetry, ultrasonic telemetry or other telemetric technologies.

As used herein, “routine” refers to an established function, operation or procedure of a system, such as an embedded software module that is performed or is available to be performed by the system. Routines may be activated manually such as by an operator of a system, or occur automatically such as a routine whose initiation is triggered by another function, an elapsed time or time of day, or other trigger. The devices, apparatus, systems and methods of the present invention may include or otherwise have integrated into one or their components, numerous types and forms of routines. An “adaptive processing routine” is activated to determine and/or cause a routine or other function to be modified or otherwise adapt to maintain or improve performance. A competitive routine is activated to provide a competitive function for the patient of the present invention to compete with, such as a function which allows an operator of the system to compete with the patient in a patient training task; or an automated system function which controls a visual object which competes with a patient controlled object. A “configuration routine” is activated to configure one or more system configuration parameters of the system, such as a parameter that needs an initial value assigned or a parameter that needs an existing parameter modified. A system “diagnostic routine” is activated, such as automatically or with operator intervention, to check one or more functions of the system to insure proper performance and indicate acceptable system status to one or more components of the system or an operator of the system. A “language selection routine” is activated to change a language displayed in text form on a display and/or in audible form from a speaker. A “patient training routine” is activated to train the patient in the use of the system and/or train the system in the specifics of the patient, such as the specifics of the patient's multicellular signals that can be generated by the patient and detected by the sensor. A “permission routine” is activated when a system configuration or other parameter is to be initially set or modified in a secured manner. The permission routine may use one or more of: a password; a restricted user logon function; a user ID; an electronic key; a electromechanical key; a mechanical key; a specific Internet IP address; and other means of confirming the identify of one or more operators prior to allowing a secure operation to occur. A “remote technician routine” is activated to allow an operator to access the system of the present invention, or an associated device, from a location remote from the patient, or a system component to be modified. A “system configuration routine” is activated to configure the system, or one or more components or associated devices of the system. In a system configuration routine, one or more system configuration parameters may be modified or initially set to a value. A “system reset routine” is activated to reset the entire system or a system function. Resetting the system is sometimes required with computers and computer based devices such as during a power failure or a system malfunction.

General Description of the Embodiments

Systems, methods, apparatus and devices consistent with the invention detect cellular signals generated within a patient's body and implement signal processing techniques to generate processed signals for transmission to one or more devices to be controlled. The systems include a biological interface apparatus that allows the patient voluntary control or physiology control of a controlled device. The systems further include a joint movement device including devices that move one or more joints of a patient, such as a powered exoskeleton device or a Functional Electrical Stimulation (FES) device, and a device that moves a joint of a prosthetic limb for an amputee patient. Data transferred from the joint movement device and/or data transferred regarding the joint movement device, to one or more components of the system, improves control, safety and reliability of cellular signal control of joint movements.

The biological interface apparatus includes a sensor, comprising a plurality of electrodes that detect multicellular signals from one or more living cells, such as from the central or peripheral nervous system of a patient. The biological interface apparatus further includes a processing unit that receives and processes the multicellular signals and transmits a processed signals to a controlled device. The processing unit utilizes various electronic, mathematic, neural net and other signal processing techniques in producing the processed signal. System data, such as joint movement device data, can be used in one or more calculations such as the transfer function used to produce the processed signals.

Also disclosed is a joint movement device including a force translating structure attached to one or more portions of a patient. A force producing assembly applies forces to one or more cables attached to the force translating structure, transferring a resultant force to one or more of the patient's joints. In an alternative embodiment, a torque generating assembly is included, applying controllable torque to the elbow of the patient, wherein the force translating structure is attached to the patients fingers and/or wrist. In a system configuration, further included is a biological interface apparatus that includes a sensor that detects multicellular signals and a processing unit that processes the multicellular signals to produce processed signals. These processed signals are used by the system to control either or both the force producing assembly and the torque generating assembly.

### DETAILED DESCRIPTION OF THE EMBODIMENTS

Reference will now be made in detail to the present embodiments of the invention, examples of which are illustrated in the accompanying drawings. Wherever possible, the same reference numbers will be used throughout the drawings to refer to the same or like parts.

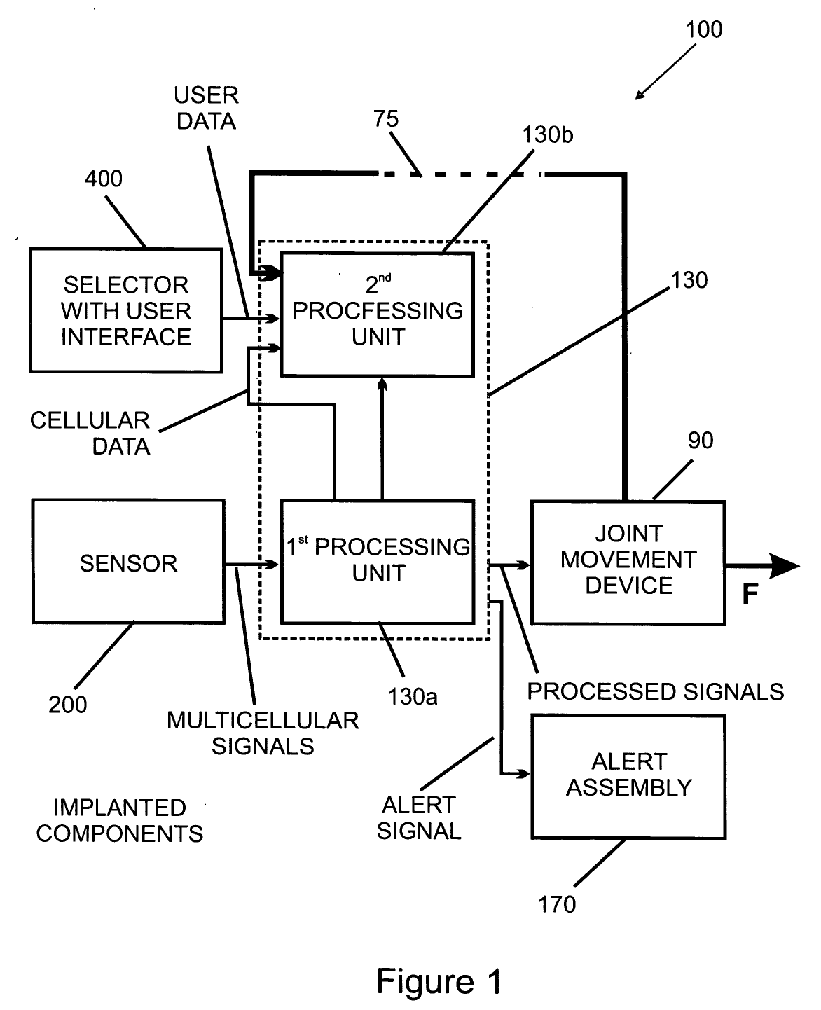

Referring now to FIG. 1, a schematic representation of a preferred embodiment of a joint movement device being controlled by a biological interface apparatus of the present invention is illustrated. The biological interface apparatus includes a sensor 200 for collecting multicellular signals of a patient. Sensor 200 includes multiple electrodes that allow for detection, such as chronic detection of these multicellular signals, these electrodes preferably implanted in the brain of the patient, but potentially implanted in one or more locations that allow detection of signals such as neural signals and other cellular activity. Sensor 200 may assume numerous forms such as an array of electrodes connected to a base or substrate or wire or wire bundle electrodes. Sensor 200 may consist of a single component, or multiple discrete components such as a component with electrodes implanted in, on or near the brain, and a component implanted in, on or near the spinal cord.

Biological interface apparatus 100 further includes processing unit first portion 130a, which in combination with processing unit first portion 130b comprises processing unit 130, which receives the multicellular signals from sensor 200, and processes these multicellular signals to produce processed signals, which are used as a control signal to be sent to a controllable device. Processing unit first portion 130a and sensor 200, connected with a multi-conductor bundle of wires, are both implanted under the skin of the patient. Processing unit first portion 130a includes means of converting the processed signals into digital information or data. This cellular data can then be transmitted wirelessly through the skin, such as via infrared wireless communication means, to processing unit second portion 130b. The multicellular signals are converted to digital information using multiple electronic components used to buffer the signal, amplify the signal, perform an analog to digital conversion, and other signal processing functions. Processing unit first portion 130a may include an integral power supply, such as a power supply rechargeable via inductive coils also integral to processing unit first portion 130a. The power supply may include a rechargeable battery, or a capacitive storage bank, supplying power to the one or more implanted electronic components requiring energy to operate.

The biological interface apparatus 100 further includes a controlled device, joint movement device 90 which receives the processed signals from processing unit 130, either from processing unit first portion 130a, processing unit second portion 130b or both. Joint movement device has numerous pieces of information associated with it, joint movement device data, which may correspond to one or more mechanical, electrical, or other parameters of the joint movement device. The joint movement device data may be known prior to its use, such as: one or more time constants; range of motion values; required power or energy levels; other boundary condition information; other manufacturer supplied information; and other joint movement device configuration parameters which may be used by processing unit 130 to produce the processed signals used to control joint movement device 90. Configuration parameters may include: maximum extension of a joint movement device, minimum or maximum angle of a controlled joint, minimum or maximum velocity or acceleration of a controlled joint, minimum or maximum torsional force to be applied, and combinations thereof. The joint movement device data may be transmitted to avoid damage to joint movement device 90, or joint movement device 90 attempting to enter an improper state, such as via an inappropriate control signal transmitted to the joint movement device 90 by processing unit 130. The joint movement device data may be gathered some time after or during its use, such as: information from one or more sensors integral to joint movement device 90 such as position sensor, contact sensor, force pattern sensor or sensor; stress sensor, strain gauge, pressure sensor, vertical position or tilt sensor, energy sensor such as voltage or current sensor and temperature sensor; energy dissipation information; historic use information such as performance information including error or alarm condition information; configuration information such as calibration information; and other information generated during the use of joint movement device 90.

Joint movement device data, as well as potentially other data, is transmitted from joint movement device 90 to processing unit second portion 130b, via wireless transmissions 75. In an alternative embodiment, wired conduits are incorporated between joint movement device 90 and processing unit second portion 130b. This data from joint movement device 90, as well as other joint movement device data received by processing unit 130, is used one or more components of processing unit 130 to determine a system configuration parameter value, hereafter synonymous with system configuration parameter. These system configuration parameters are preferably used to produce a transfer function to apply to the multicellular signals of sensor 200 to produce the processed signals transmitted to joint movement device 90. In an alternative embodiment, joint movement device 90 includes a visible bar code, and a bar code reader in communication with processing unit 130 uploads the bar code information to processing unit 130.

Joint movement device 90 can take numerous forms, including a device to move a patient's joint such as an FES device with implanted FES stimulators or a powered exoskeleton device. Alternatively joint movement device 90 may include a prosthetic limb, the force generated applied to one or more artificial joints of the processed limb. Combinations of FES devices, exoskeleton devices and prosthetic limbs can be used, with one or more controlled by the processed signals of processing unit 130, to restore motor function of a patient such as a quadriplegic patient, paraplegic patient, and/or an amputee. The patient joints that can be controlled, such as via an FES device or an exoskeleton, include but are not limited to: a shoulder; an elbow; a wrist; a finger joint; a hip; a knee; an ankle; a toe joint; a metacarpophalangeal joint; an interphalangeal joint; a temporomandibular joint; and combinations thereof. The artificial or prosthetic limbs that can be controlled include but are not limited to: foot; leg without knee; leg with knee; hand; arm without elbow; arm with elbow; and combinations thereof.

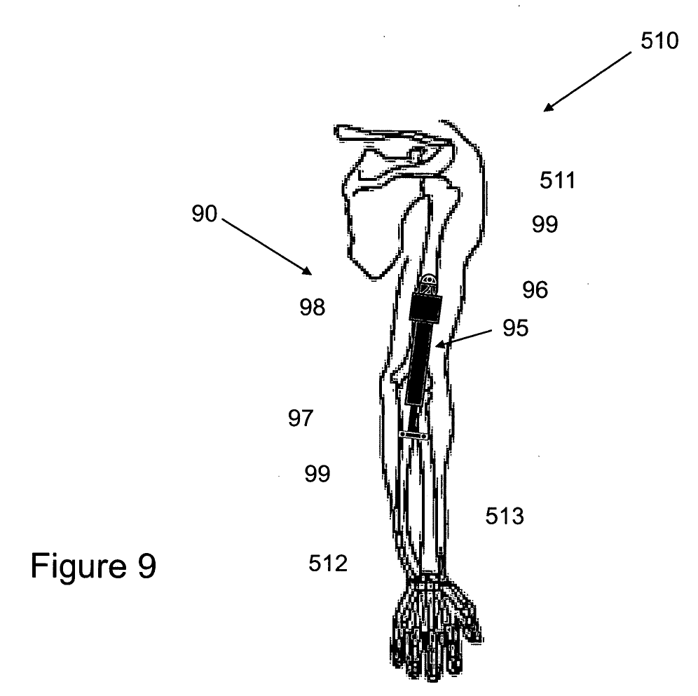

Joint movement device 90 includes a force generator, to directly or indirectly apply a force to a joint of the patient or a joint of a prosthetic device. The force generator is selected from the group consisting of: a motor; a linear actuator; a solenoid; a servo; an electromagnet; a Nitinol™ wire; a fluid pump such as a hydraulic pump; an air pump such as a pneumatic pump; and combinations thereof. In addition, joint movement device 90 preferably includes a mechanical advantage assembly, such as to increase the force generated, while reducing a distance such as an angular distance traveled, or to increase an angular distance, while decreasing the force generated. The mechanical advantage assembly includes one or more of: a lever arm; a cam; a pneumatic assembly; and a hydraulic assembly. The force generated by the various assemblies may result in a torsional force or a linear force. While the exoskeleton device and the prosthetic limbs are attached to the patient, the FES device and other joint movement devices are implanted or at least partially implanted within the patient. In one embodiment of the joint movement device, described in detail in reference to FIG. 9, a controllable piston assembly is implanted in the patient, wherein a housing is attached to a first a first bone of the patient and a advanceable and retractable piston is attached to a second bone of the patient, such that the advancement and retraction of the piston applies a force to the patient joint connecting the first and second bones of the patient.

Joint movement device data can be transmitted on a planned periodic schedule, on a request for transmission by processing unit 130 or other system component, or upon another trigger such as a specific condition detected by one or more sensors integral to joint movement device 90. An analysis of the joint movement device data received by processing unit 130, either from joint movement device 90 or another source, may trigger a change to one or more configuration parameters of biological interface apparatus 100 to change, such as a parameter change that causes a state of the system to change. In a preferred embodiment, the gain of the signal sent to the force generator changes, such as the gain sent to one or more of a: motor; linear actuator; solenoid; servo; electromagnet; pneumatic pump; hydraulic pump; and Nitinol wire. In another preferred embodiment, the limit angle, such as the maximum or minimum angle of a joint, such as a boundary condition for a patient joint or prosthetic limb joint, is modified to improve control of the joint movement device.

The biological interface apparatus of FIG. 1 further includes alarm assembly 170. Based on analysis of one or more pieces of data, such as joint movement device data, processing unit 130 will transmit an alert signal to alert assembly 170 to trigger an alert event. An alert event preferably includes an audible alert transducer that is activated to notify the patient and/or people in the relative vicinity of the patient such as a family member or health care provider. In an alternative or additional embodiment, alert assembly 170 further includes a telephone access function, such as a cellular telephone function which when receiving a specific alert signal will dial one or more predetermined numbers, such as 911 or a number to contact a caregiver or family member, and deliver one or more predetermined messages such as a distress message including the location of the patient, such as a location determined by a GPS assembly integral to the biological interface apparatus 100.

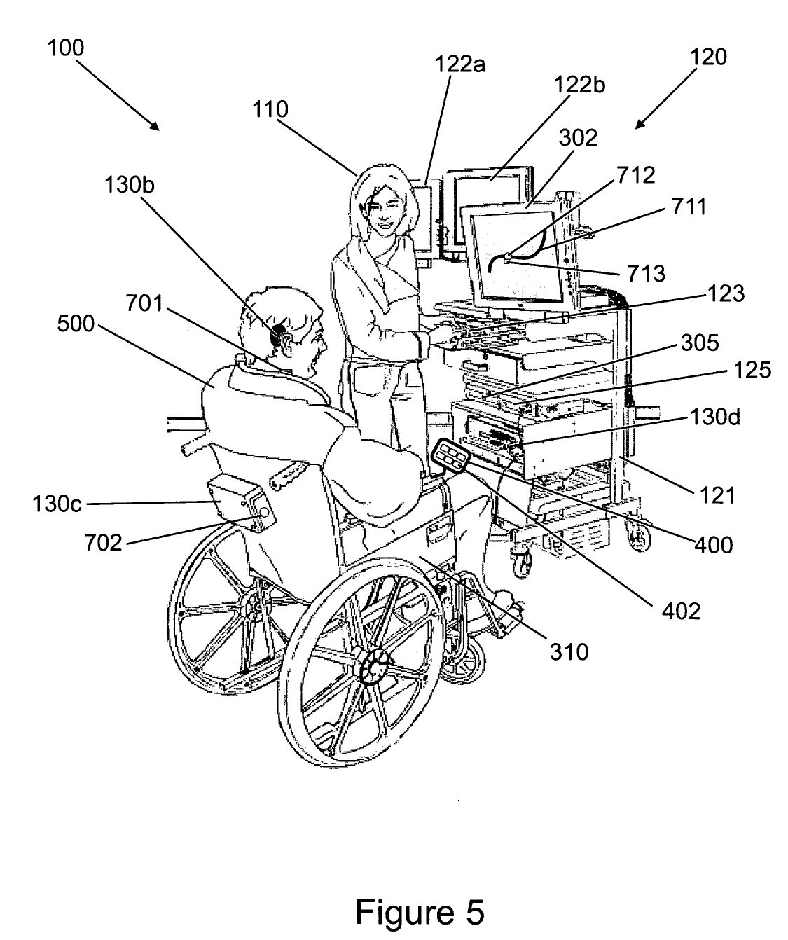

As stated hereabove, in addition to transmissions from joint movement device 170, joint movement device data can be received by processing unit 130 from other means, such as via an operator utilizing a user interface incorporated into selector module 400. Selector module 400 may include a touch screen display that allows input of joint movement device data, such as device type, model, and other user data that may be used by processing unit 130 to modify one or more system configuration parameters, such as parameters used to create the processed signals transmitted to joint movement device 90. In addition, selector module 400 is used by an operator, such as the patient, a physical therapist, or other operator of the system, to select or change which controlled device is to be controlled, such as joint movement device 90 or a separate controlled device, not shown, but described in detail in reference to FIG. 5 herebelow.

The one or more transmissions of joint movement device data of the devices and apparatus of the present invention can be initiated, or triggered, by an operator intervention such as a data entry made to selector module 400, or automatically by an apparatus component. An operator may enter data as a result of a physical therapy session, or event, conducted with the patient. In a preferred embodiment, a physical therapy event generates patient range of motion data applicable to the joint movement device. In another preferred embodiment, patient feedback to the physical therapist, such as indication of positions to avoid due to real pain or phantom pain, is joint movement device data transmitted to processing unit 130 via selector module 400. In another preferred embodiment, the joint movement device data is transmitted to avoid patient spasticity, such as positions or movements that caused patient spasticity in a physical therapy session. Processing unit 130 may apply one or more safety factors, such as a safety factor applied to a range of motion, to avoid patient discomfort. Joint movement device data can also be input by an operator at a remote location, such as an operator that transmits information over a computer network such as the Internet, the network in electronic communication with a component of apparatus 100. Joint material device data and its transmission can be triggered by one or more system configuration procedures that are conducted automatically by the system of by an operator. In a preferred embodiment, a calibration procedure, such as a joint movement device calibration, causes multiple pieces of joint movement device data to be generated and transmitted to processing unit 130. A calibration performance test, such as a test that results in inadequate or failed performance, may generate data to eliminate the failure. A patient training procedure, such as one utilizing the joint movement device 90, may also generate joint movement device data, again such as to improve performance or eliminate an unacceptable condition.

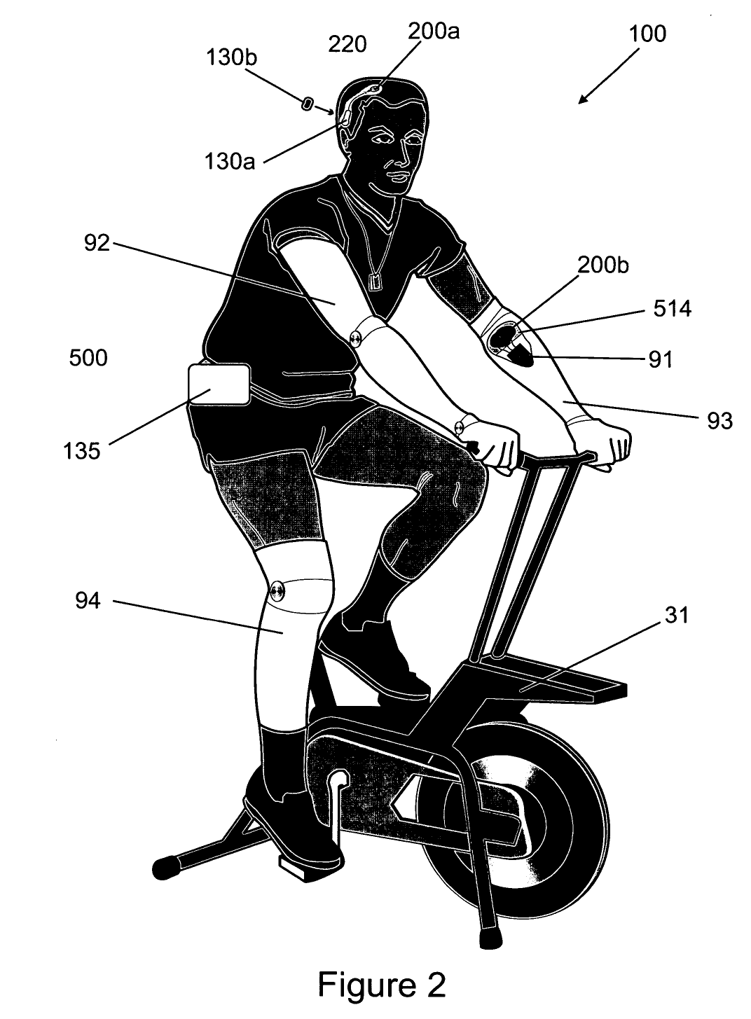

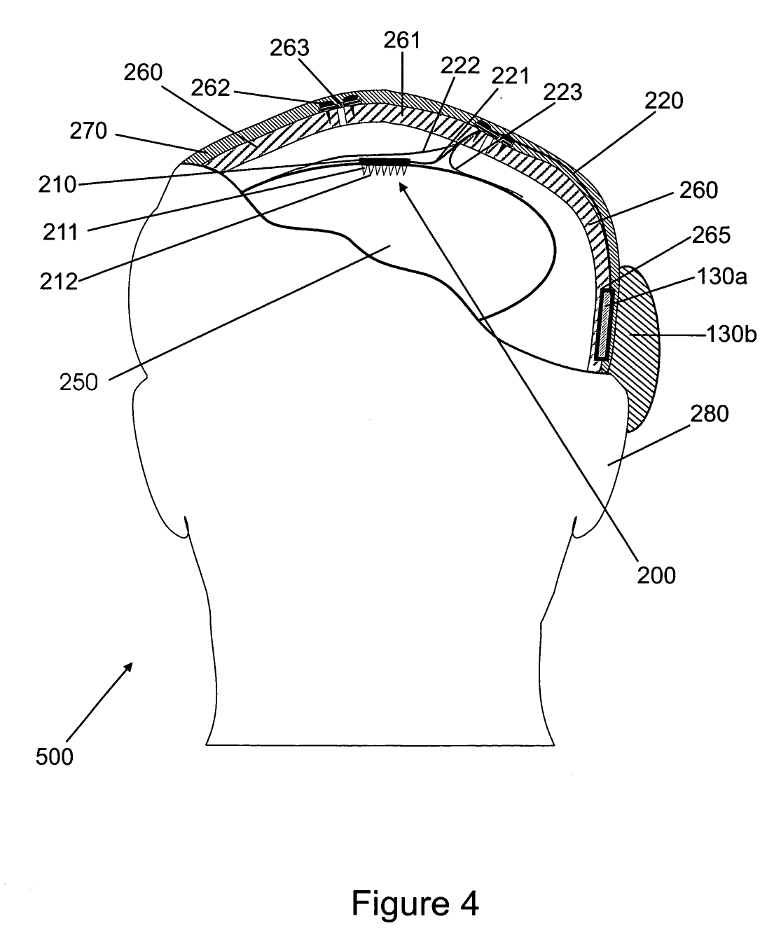

Referring now to FIG. 2, a biological interface apparatus for collecting multicellular signals emanating from one or more living cells of a patient and for transmitting processed signals to a joint movement device is illustrated. Biological interface apparatus 100, which is described in detail in reference to FIG. 4 and FIG. 5 herebelow, includes first sensor 200a and a processing unit for processing the multicellular signals that are detected by first sensor 200a. The processing unit comprises two discrete components, processing unit first portion 130a that is implanted under the scalp of patient 500, and processing unit second portion 130b that is external to the patient. First sensor 200a is illustrated through a view of the skull that has been cutaway, first sensor 200a being implanted in the motor cortex of patient 500's brain. In a preferred embodiment, sensor 200a is implanted in a portion of the motor cortex associated one or more the joints or surrogate, prosthetic joints controlled by one or more joint movement devices of apparatus 100. In a preferred embodiment, a functional MRI (fMRI) is performed prior to the surgery in which the patient imagines moving one or more target joints, and first sensor 200a is located based on information output from the fMRI. A wire bundle 220 connects first sensor 200a to processing unit first portion 130a, which has been placed in a recess, surgically created in patient 500's skull, viewed in FIG. 1 through a cutaway of patient 500's scalp. Wire bundle 220 includes multiple, flexible insulated wires, preferably a single wire for each electrode. In an alternative embodiment, one or more single wires carry cellular signal transmissions from two or more electrodes. The surgical procedure required for the implantation of wire bundle 220, as well as first sensor 200a and processing unit first portion 130a, is described in detail in reference to FIG. 4 herebelow. Alternatively or additionally, a cellular signal sensor component may be placed in numerous locations such as the spinal cord or a peripheral nerve.

Processing unit first portion 130a transmits data, such as with RF or infrared transmission means, to a receiver of processing unit second portion 130b, which is shown as in the process of being removably placed at a location near the implant site of processing unit first portion 130a. In a preferred embodiment, magnets integral to either or both processing unit discrete components are used to maintain the components in appropriate proximity and alignment to assure accurate transmissions of data. One or more patient input devices, all not shown, may be affixed to patient 500 such as: chin joystick; EEG activated switch such as the switch manufactured by BrainFingers of Yellow Springs, Ohio, USA; eyebrow switch such as an eyebrow EMG switch manufactured by Words+Inc. of Lancaster, Calif.; eye tracker such as the device manufactured by LC Technologies of Fairfax, Va., USA; a head tracker such as the device manufactured Synapse Adaptive of San Rafael, Calif., USA; neck movement switch; shoulder movement switch; Sip n' Puff joystick controller such as the controller manufactured by QuadJoy of Sheboygan, Wis., USA; speech recognition switch; tongue switch such as a tongue palate switch; and combinations thereof. These switches are used to provide a patient activated input signal to biological interface apparatus 100. In an alternative or additional embodiment, one or more of these switches are used to provide a patient activated input to one or more components of apparatus 100. Patient input switches incorporated into one or more apparatus, device, methods and systems of the present invention can be used in the performance of various system functions or routines and/or to initiate various system functions or routines. In a preferred embodiment, a patient input switch is used to change the state of the system, such as when the system state changes to: a reset state; the next step of a configuration routine, a stopped state; an alarm state; a message sending state, a limited control of controlled device state; and combinations thereof. Alternative to the patient input switch is a monitored biological signal that is used for a similar change of state function. Applicable monitored biological signals are selected from the group consisting of: eye motion; eyelid motion; facial muscle activation or other electromyographic activity; heart rate; EEG; LFP; respiration; and combinations thereof.

Patient 500 is a patient with multiple lost limbs, common to soldiers returning from the Iraq war of these early 2000's. Patient 500 of FIG. 2 has received multiple joint movement devices of the present invention including arm prosthetic 92, forearm prosthetic 93 and lower leg prosthetic 94. Each of these prosthetics includes integral power supplies such as rechargeable batteries, force generating assemblies such as motors and gear assemblies attached to hinges and other joints within the prosthetic, and wireless transceivers for sending and receiving data to or from the other prosthetics, processing unit second portion 130b and/or other apparatus 100 discrete components. The integral power supplies are preferably rechargeable, and may supply power to additional components of apparatus 100, such as a separate joint movement device. Each of the prosthetics may include attachment to a second sensor, such as forearm prosthetic 93, which is connected to second sensor 200b. Sensor 200b, preferably wire or wire bundle electrodes but potentially an array of projections with one or more electrodes along the projections length, is implanted within arm stump 514 of the left arm of patient 500 and is in proximity to one or more nerves that previously were in neurological communication within the one or more muscles in the portion of patient 500's left arm that is now missing. The cellular signals received from second sensor 200b are transmitted by electronic module 91 of forearm prosthetic 93 to processing unit second portion 130b, which, in combination with the cellular signals received from first sensor 200a are processed by the processing unit to produce processed signals. Electronic module 91 may further include one or more computational means and other signal processing functions such as to pre-process the data sent to processing unit second portion 130b, and to post-process the processed signals received from processing unit second portion 130b. Electronic module 91, as well as electronic modules integral to arm prosthetic 92 or leg prosthetic 94, may receive data from other types of sensors, such as one or more sensors monitoring a physiologic parameter of the patient, a performance parameter of the prosthetic or an environmental parameter.

The prosthetic devices of FIG. 2 are all controlled devices and joint movement devices of the present invention, or alternatively include a component that is a controlled device and/or joint movement device of the present invention. In an alternative embodiment, one or more of prosthetic 92, prosthetic 93, and prosthetic 94 may be controlled internally or otherwise by means other than the processed signals of biological interface apparatus 100. In another alternative embodiment, one or more exoskeleton devices and/or FES devices can be utilized with patient 500 of FIG. 2, as controlled devices of apparatus 100 or otherwise. Additional, not joint moving controlled devices may also be controlled by the processed signals of apparatus 100 such as vehicles including: a motorized scooter, a wheelchair, a car, a boat, an aircraft, and combinations thereof.

In a preferred embodiment, data is transmitted from prosthetic device 92, prosthetic device 93 and/or prosthetic device 94 to processing unit second portion 130b or another component of apparatus 100, such that an analysis of this data can be used to set, modify, and/or create a system configuration parameter. These system configuration parameters may be a parameter of a joint movement device or other component of apparatus 100. Joint movement device parameters can be related to one or more boundary conditions of a joint movement device such as: maximum extension, minimum or maximum angle, minimum or maximum velocity or acceleration such as angular velocity or angular acceleration, minimum or maximum force such as torsional force, range of motion limits, and combinations thereof.

Other components of biological interface apparatus 100 may transfer data, such as joint movement device data, to a separate component of apparatus 100, such as processing unit second portion 130b, such information used to modify one or more system configuration parameters such as a parameter used in a transfer function to produce the processed signals sent to one or more controlled devices. Patient 500 uses exercise bike 31 to perform a physical therapy session or event, and bike 31 may include one or more sensors, or otherwise provide data that relates to the joint movement device or other apparatus component. Other forms of physical therapy apparatus such as stair machines, weight machines, patient joint angle measuring devices, torque measurement devices, and other related physical therapy equipment may provide data, such as via integral sensors, that is used by apparatus 100 to modify one or more configuration parameters. These data can be transmitted via wired or wireless means, or may provide the data to an operator, such as via a visual display, who then enters the data manually into a component of apparatus 100.

Another component of the apparatus 100 of FIG. 2 that transmits joint movement device data and other data is interface device 135. In a preferred embodiment, interface device 135 includes a user interface, not shown but preferably a touch screen display, such that data can be entered by, or communicated to, an operator. Interface 135 includes a power supply, such as a rechargeable or replaceable battery, and may supply power to one or more components of apparatus 100. Interface 135 includes wireless transmission and receiving means, such as an RF transceiver, and can send and receive information to or from each prosthetic device. Interface 135 is attached to a physiologic sensor, not shown but preferably an EKG lead attached to the patient, such that the physiologic information can be fed back from interface 135 to one or more components of the processing unit of apparatus 100, and the patient's heart rate data can be used in one or more analyses, such as a safety routine which alters the processed signals when the patient's heart rate is at an unacceptable state. Interface 135 includes electronic components to perform computational and other signal processing functions and may include a portion of the processing unit of the present invention, as well as perform as a patient input device for patient 500. In an alternative embodiment, one or more of arm prosthetic 92, forearm prosthetic 93 and/or leg prosthetic 94 includes a portion of the processing unit of apparatus 100. Interface 135 preferably provides means of activating and deactivating one or more controlled devices such as the joint movement devices of the present invention.

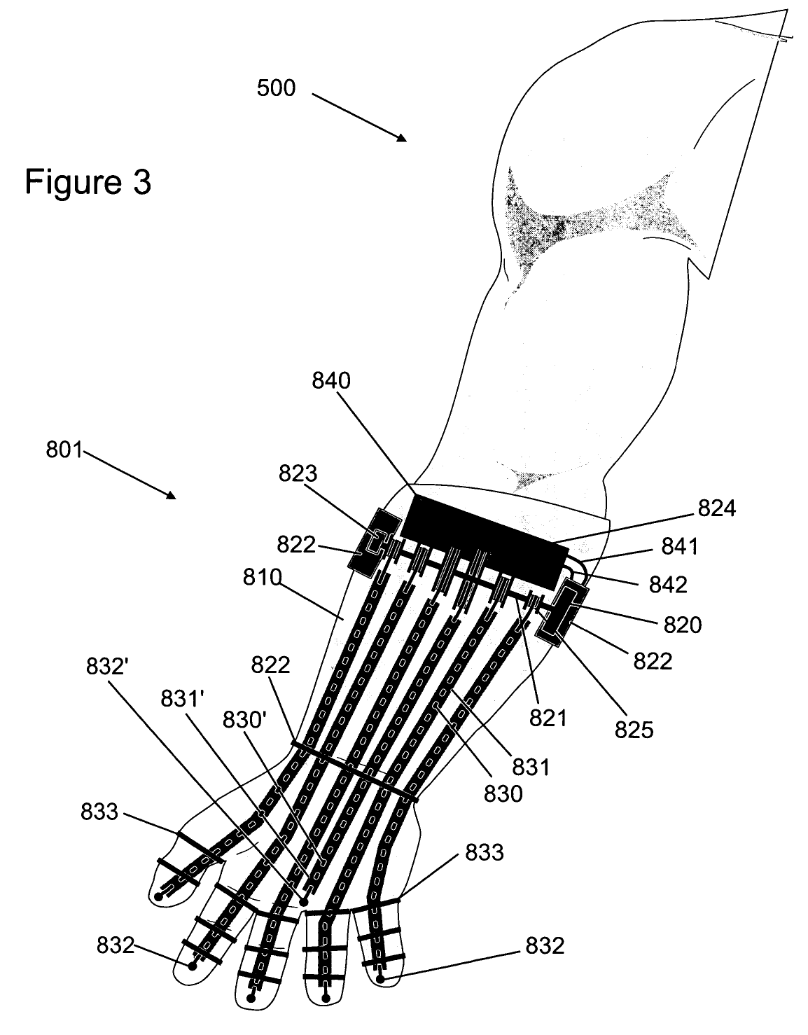

Referring now to FIG. 3, a preferred embodiment of the joint movement device of the present invention is illustrated, wherein the joint movement device is for applying a force to one or more joints of the patient. The joint movement device, hand controlling glove assembly 801, shown from the palm side orientation, is placed over the hand a patient 500, such that lost or compromised control of patient 500's hand and wrist can be restored. Assembly 801 is configured to open, close and partially close the fist, such as to grasp an object, as well as independently control the fingers of patient 500. In addition, assembly 801 can be used to precisely flex the wrist of patient 500. Glove 810, an elastic, flexible material such as an elastic fabric, acts as a force translating structure, glove 810 being in close contract with multiple portions of patient 500 from the elbow to the tips of one or more fingers. In a preferred embodiment, glove 810 substantially surrounds, such as making contact with more than half the skin surface area from the proximal end to the distal end of glove 810, the end of the elbow to the tip of the fingers respectively.

Attached to the palm side of glove 810 are multiple longitudinal coverings, such as longitudinal coverings 831 and 831′. Each longitudinal covering 831 has a proximal end near the proximal end of glove 810, covering 831 extending distally to a location on the fingertip portion of glove 810. Longitudinal covering 831′ has a proximal end near the proximal end of glove 810 and extends distally to a location on the palm portion proximate the first joint of the middle finger joint portion of glove 810. Longitudinal coverings 831 and 831′, preferably made of a material less flexible than the material of glove 810, are fixedly attached along their edges to glove 810, such that a tunnel is created from the proximal end to the distal end of covering 831. The attachment means may include a fabric adhesive or a stitching along the edges, adhesive or stitching not shown. Control cables 830 and Control cable 830′ are slidingly received by the tunnels created between longitudinal coverings 831 and 831′ respectively, the attachment means configured such that each control cable remains in close proximity to glove 810 as sufficient tension to be transmitted to one or more joints is applied to each controlled cable. Control cables 830 and 830′ each have a proximal end and a distal end, and are preferably constructed of a flexible material with limited stretch such as a fluorocarbon fishing line such as Bass Pro Shops XPS Signature Series Fluorocarbon Fishing Line from Bass Pro Shops of Springfield, Mo. Other flexible conduits including other monofilament fishing lines, wires, and superelastic metals such as Nitinol wires.

The distal end of control cables 830 are fixedly attached to each finger tip of glove 810 at fixation point 832. The distal end of control cable 830′ is fixedly attached to fixation point 832′ on the palm portion of glove 810 near the first joint of the middle finger. The proximal ends of control cables 830 and control cable 830′ is operably attached to a pulley, such as large pulley 824 and small pulley 825. Each pulley is engaged to an axle, axle 821 that is controllably rotated by motor assembly 820. In a preferred embodiment, one or more axles 821 can be controllably disengaged and re-engaged with axle 821, such as via an electronic brake or clutch which receives power and signals from one or more slip rings, all not shown, such that one or more pulleys can be independently rotated utilizing a single motor driven axle. Rotation of each pulley in the proper direction causes the operably attached control cable to retract. Proper rotation of large pulley 824 causes control cable 830′ to retract, slidingly retracting within the tunnel formed between glove 810 and covering 831′. A resultant force is applied at fixation point 832′ such that a force is applied to the wrist of the patient tending the wrist to flex in an inward direction. Proper rotation of small pulley 825 causes its control cable 830 to retract, slidingly retracting within the tunnel formed between glove 810 and its associated covering 831. A resultant force is applied at its associated fixation point 832 at the tip of the little finger of glove 810 such that a force is applied to the little finger of the patient tending each of the joints of the little finger to flex inwardly.

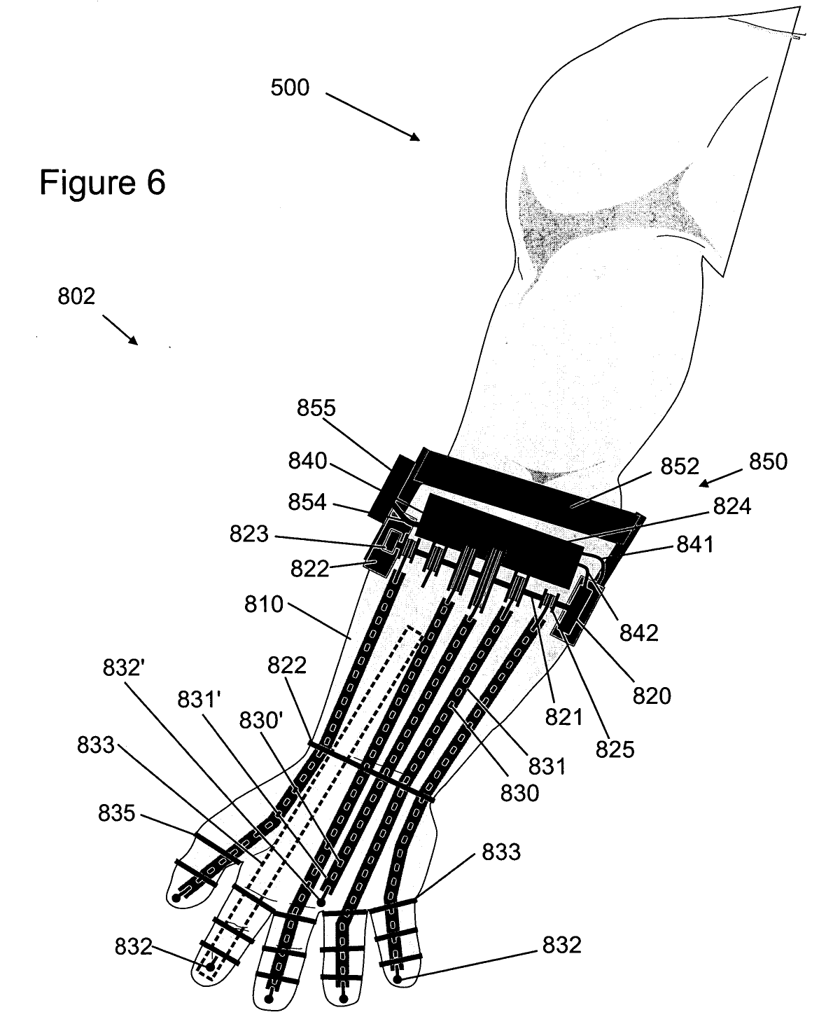

If patient 500 has received assembly 801 to improve gripping force or otherwise improve compromised motor function of the hand and/or wrist, patient 500 may straighten the wrist and/or fingers, such as when the pulleys have been disengaged from axle 821, causing the pulleys to rotated to allow the corresponding control cable to advance. In an alternative embodiment, such as when patient 500 has minimal or no control of the wrist or finger joints, an elongate, resiliently elastic member, not shown but shown and described in reference to FIG. 6, such as a long thin strap of flexible metal such as spring steel or superelastic alloy is used to resiliently bias the wrist or finger in a straight position. The resiliently elastic member is fixedly attached to the dorsal side of the glove and positioned across the appropriate joint or joints to cause the wrist and/or finger to be biased in a relatively straight configuration, such as via a straight or slightly curved configuration. After an engaged pulley is rotated such that a control cable is retracted and force applied to the fixation point, the wrist or one or more finger joints can have a force applied to tend the one or more joints to curl inward, toward the flexor muscles on the underside of the patient's forearm, overcoming the forces applied by the resiliently elastic member. If motor assembly 820 maintains the position of axle 821, a resultant force will remain at the associated fixation point, maintaining the one or more joints with an applied force tending them to curl inward. When motor assembly 821 places axle 821 in a free spinning or low torque configuration and/or the associated pulley disengages from axle 821, the resiliently elastic member will cause the associated joint to tend toward a straightened state.

Axle 821 has a proximal end attached to motor assembly 820 and a distal end which is rotationally received by bearing 823 such that the radial loads applied by the control cables 830 and 830′ result in minimal frictional loss. Bearing 823 and motor assembly 820 are each fixedly mounted to mounts 822, and each of mounts 822 are fixedly mounted to glove 810. Motor assembly 820 receives power and control signals from electronic module 840, a module including multiple functions such as: a power supply such as a rechargeable battery, a wireless transceiver for sending and receiving data, such as receiving the processed signals of the present invention transmitted by a processing unit of a biological interface apparatus, computational and other signal processing circuitry and functions, one or more sensor functions such as a sensor that monitors tension in one or more control cables or a power level of a power supply, and other functions. Electronic module 840 is electrically connected to motor assembly 820 via wiring 851 and wiring 842, such wiring including power and control signals.

Motor assembly 820 includes one or more rotational actuators such as rotational solenoids and rotational motors. Various types of rotational motors can be integrated such as a stepper motor, a DC motor, an AC motor, a synchronous motor, and combinations thereof. In a preferred embodiment, a stepper motor is used wherein the holding, detent force is chosen to prevent rotation of the axel without a drive signal and power being applied to the stepper motor. Detent force, also referred to as residual torque or holding torque, is the force or torque present in an unenergized stepper motor caused by its magnetic rotor. Due to the detent torque, stepper motors tend to hold their position even when unenergized. In a preferred embodiment, motor assembly 820 includes a position encoder, such as an optical encoder used to accurately provide feedback proportional to axle position and or angular displacement to provide precise control and/or detect a malfunction. Motor assembly 820 includes a mechanical advantage assembly, such as an assembly including one or more gears, cams or lever arms. In an alternative embodiment, motor assembly 820 includes a linear actuator, such as a solenoid or a shaped memory alloy wire such as a Nitinol wire. While motor assembly 820 receives power from electronic module 840, motor assembly 820 may include an integral power supply, such as a rechargeable battery.

Also depicted in the hand controlling glove assembly 801 of FIG. 3 are constraining bands 833 located proximate each joint of patient 500's hand and wrist such that as the control cables 830 and 830′ are placed in tension, flexion is directed at the locations of the constraining bands to more closely approximate normal forces applied to a healthy individual. Bands 833 are constructed of material to have minimal stretch, such as a similar material used for control cables 830.

Hand controlling glove assembly 801 can be used to move, such as a rotation, one or more joints or to put a joint in tension, such as to push against a surface including the grasping an object with one or more finger joints. In a preferred embodiment, hand controlling glove assembly 801 is a controlled device of the biological interface apparatus of the present invention, wherein electronic module 840 receives processed signals for causing individual control cables 830 and 830′ to retract, applying force to one or more joints independently. It should be noted that the biological interface of the present invention is unique in its ability to provide a sophisticated control signal enabling patient 500 to cause joint movement similar to normal hand, wrist and other joint control. The sensor of the biological interface apparatus can be placed in the portion of the brain's motor cortex associated with the joints to be controlled, or proximate to one or more nerves of the central or peripheral nervous system associated with the specific joints.

Glove 810 can take numerous forms, such as complete skin coverage, to selected coverage at or around specific joints. In an alternative embodiment, glove 810 may have fixedly attached to it a flexible battery, not shown, such as a flexible battery manufactured by Cymbet Corporation of Elk River, Minn., USA.

Hand controlling device assembly 801 preferably includes one or more sensors, not shown, these sensors working with signal processing electronics of electronic module 840. The sensors can be used to provide data related to one or more of: force feedback, tension in a cable, energy measurement such as a current or voltage measurement, a pressure measurement, a stress measurement, a strain measurement, and combinations of the preceding. In a preferred embodiment, the motor assembly stops retraction of one or more control cables 830 or 830′ when a signal or processed signal from a sensor surpasses a threshold, such as an adjustable threshold.

While the longitudinal coverings 831 and 831′ are shown as a long piece of material extending from a location proximate the elbow to a location on the hand, in an alternative embodiment, multiple short pieces of material, not shown, create multiple individual tunnels between the material and glove 810, similarly maintaining the captured control cable 830 or 830′ in close proximity to glove 810 when the control cable is under tension. In an alternative embodiment, the coverings are located on the dorsal side of glove 810, such that rotation of a pulley causes the operably attached control cable to cause one or more joints to straighten, such as from a curved condition. In this alternative embodiment, curved, resiliently biased members can be placed on the palmar side of glove 810 such that a wrist joint and/or one or more finger joints is resiliently biased in a curved state.

While the joint movement device of FIG. 3 is attached to the hand and wrist of patient 500, it should be understood that similar constructions could be applied to a different joint, or different set of joints, such as the ankle and toes of patient 500 wherein the force translating structure takes on a sock-like construction. For joints with multiple degrees of freedom, such as an ankle joint, shoulder joint, wrist, finger joint and hip joint, multiple control cables can be placed across the joint, but on different sides of the joint, to cause flexion in the direction on which the control cable is placed. In applications for wrist flexion, a control cable is placed across the wrist proximate the middle of the palm and across the wrist on the ulnar side of the hand. Resiliently biased members as have been described hereabove, may be placed on the sides opposite the control cable positions such as to generate a torque in the opposite direction. In an alternative embodiment, additional control cables are used instead of the resiliently biased members, such that retraction of a first control cable causes the associated joint to tend to curl or straighten in a first direction, and retraction of a second control cable causes the associated joint to curl or straighten in the opposite direction.

Referring now to FIG. 4, a brain implant apparatus consistent with an embodiment of the present invention is illustrated. As shown in FIG. 4, the system includes an array of electrodes assembly, sensor 200, which has been inserted into a brain 250 of patient 500, through a previously created opening in scalp 270 and skull 260 in a surgical procedure known as a craniotomy. Sensor 200 includes a plurality of longitudinal projections 211 extending from a base, array substrate 210. Projections 211 may be rigid, semi-flexible or flexible, the flexibility such that each projection 211 can still penetrate into neural tissue, potentially with an assisting device or with projections that only temporarily exist in a rigid condition. Sensor 200 has been inserted into brain 250, preferably using a rapid insertion tool, such that the projections 211 pierce into brain 250 and sensor substrate 210 remains in close proximity to or in light contact with the surface of brain 250. At the end of each projection 211 is an electrode, electrode 212. In alternative embodiments, electrodes can be located at a location other than the tip of projections 211 or multiple electrodes may be included along the length of one or more of the projections 211. One or more projections 211 may be void of any electrode, such projections potentially including anchoring means such as bulbous tips or barbs, not shown.

Electrodes 212 are configured to detect electrical brain signals or impulses, such as individual neuron spikes or signals that represent clusters of neurons such as local field potential (LFP) and electroencephalogram (EEG) signals. Each electrode 212 may be used to individually detect the firing of multiple neurons, separated by neuron spike discrimination techniques. Other applicable signals include electrocorticogram (ECOG) signals and other signals, such as signals between single neuron spikes and EEG signals. Sensor 200 may be placed in any location of a patient's brain allowing for electrodes 212 to detect these brain signals or impulses. In a preferred embodiment, electrodes 212 can be inserted into a part of brain 250 such as the cerebral cortex. Alternative forms of penetrating electrodes, such as wire or wire bundle electrodes, can make up or be a component of the sensor of the present invention. In addition to or alternative from neural signals, the system of the present invention may utilize other types of cellular signals to produce processed signals to control a device. The various forms of penetrating electrodes described above can be placed into tissue within or outside of the patient's cranium, such tissue including but not limited to: nerve tissue such as peripheral nerve tissue or nerves of the spine; organ tissue such as heart, pancreas, liver or kidney tissue; tumor tissue such as brain tumor or breast tumor tissue; other tissue and combinations of the preceding,

Alternatively or additionally, the sensor of the present invention may employ non-penetrating electrode configurations, not shown, such as subdural grids placed inside the cranium such as to record LFP signals. In addition to subdural grids, the sensor may consist of or otherwise include other forms of non-penetrating electrodes such as flat electrodes, coil electrodes, cuff electrodes and skin electrodes such as scalp electrodes. These non-penetrating electrode configurations are placed in, on, near or otherwise in proximity to the cells whose signals are to be detected, such as neural or other cellular signals. In another alternative embodiment, the sensor of the present invention includes detectors other than electrodes, such as photodetectors that detect cellular signals represented by a light emission. The light emission can be caused by a photodiode, integrated into the sensor or other implanted or non-implanted system component, shining one or more wavelengths of light on the appropriate cells. In addition to the numerous types of cells described above, one or more of the various configurations of the sensor of the present invention may utilize any living cell of the body that emanates cellular signals. In a preferred embodiment, the cellular signals are under voluntary control of the patient.

Although FIG. 4 depicts sensor 200 as a single discrete component, in alternative embodiments the sensor consists of multiple discrete components, including one or more types of electrodes or other cellular signal detecting elements, each configured and placed to detect similar or dissimilar types of cellular signals. Multiple sensor discrete components can be implanted entirely within: the skull, an extracranial location such as a peripheral nerve, or external to the body; or the components can be placed in any combination of these locations.

Sensor 200 serves as the multicellular signal sensor of the biological interface system of the present invention. While FIG. 4 shows sensor 200 as eight projections 211 with eight electrodes 212, sensor 200 may include one or more projections with and without electrodes, both the projections and electrodes having a variety of sizes, lengths, shapes, surface areas, forms, and arrangements. Moreover, sensor 200 may be a linear array (e.g., a row of electrodes) or a two-dimensional array (e.g., a matrix of rows and columns of electrodes such as a ten by ten array), or wire or wire bundle electrodes, all well known to those of skill in the art. An individual wire lead may include a plurality of electrodes along its length. Projections and electrodes may have the same materials of construction and geometry, or there may be varied materials and/or geometries used in one or more electrodes. Each projection 211 and electrode 212 of FIG. 4 extends into brain 250 to detect one or more cellular signals such as those generated form the neurons located in proximity to each electrode 212's placement within the brain. Neurons may generate such signals when, for example, the brain instructs a particular limb to move in a particular way and/or the brain is planning that movement. In a preferred embodiment, the electrodes reside within the arm, hand, leg or foot portion of the motor cortex of the brain. The processing unit of the present invention may assign one or more specific cellular signals to a specific use, such as a specific use correlated to a patient imagined event. In a preferred embodiment, the one or more cellular signals assigned to a specific use are under voluntary control of the patient.

Referring back to FIG. 4, the processing unit of the present invention includes processing unit first portion 130a, placed under the scalp at a location near patient 500's ear 280. Processing unit first portion 130a receives cellular signals from sensor 200 via wire bundle 220, a multi-conductor cable. In a preferred embodiment, wire bundle 220 includes a conductor for each electrode 212. Processed signals are produced by processing unit first portion 130a and other processing unit discrete components, such as processing unit second portion 130b removably placed on the external skin surface of patient 500 near ear 280. Processing unit second portion 130b remains in relative close proximity to implanted component processing unit first portion 130a through one or more fixation means such as cooperative magnetic means in both components, or body attachment means such as where the processing unit second portion 130b is attached to eye glasses, an ear wrapping arm, a hat, mechanical straps or an adhesive pad. Processing unit first portion 130a and processing unit second portion 130b work in combination to receive multicellular signal data and create a time code of brain activity.

In the preferred embodiment depicted in FIG. 4, bone flap 261, the original bone portion removed in the craniotomy, has been used to close the hole made in the skull 260 during the craniotomy, obviating the need for a prosthetic closure implant. Bone flap 261 is attached to skull 260 with one or more straps, bands 263, which are preferably titanium or stainless steel. Band 263 is secured to bone flap 261 and skull 260 with bone screws 262. Wire bundle 220 passes between bone flap 261 and the hole cut into skull 260. During the surgical procedure, bone recess 265 was made in skull 260 such that processing unit first portion 130a could be placed in the indentation, allowing scalp 270 to lie relatively flat and free of tension in the area proximal to processing unit first portion 130a. A long incision in scalp 270 between the craniotomy site and the recess 265 can be made to place processing unit first portion 130a in recess 265. Alternatively, an incision can be made to perform the craniotomy, and a separate incision made to form recess 265, after which the processing unit first portion 130a and wire bundle 220 can be tunneled under scalp 270 to the desired location. Processing unit first portion 130a is attached to skull 260 with one or more bone screws or a biocompatible adhesive, not shown.

In an alternative embodiment, processing unit first portion 130a may be placed entirely within skull 260 or be geometrically configured and surgically placed to fill the craniotomy hole instead of bone flap 261. Processing unit first portion 130a can be placed in close proximity to sensor 200, or a distance of 5-20 cm can separate the two components. Processing unit first portion 130a includes a biocompatible housing which creates a fluid seal around wire bundle 220 and numerous internal components of processing unit first portion 130a, internal components not shown. Processing unit first portion 130a internal components provide the following functions: signal processing of the cellular signals received from sensor 200 such as buffering, amplification, digital conversion and multiplexing, wireless transmission of cellular signals, a partially processed, or derivative form of the cellular signals, or other data; inductive power receiving and conversion; and other functions well known to implanted electronic assemblies such as implanted pacemakers, defibrillators and pumps.

Processing unit second portion 130b, removably placed at a location proximate to implanted processing unit first portion 130a but external to patient 500, receives data from processing unit first portion 130a via wireless communication through the skin, such as infrared or radiofrequency wireless data transfer means. Processing unit second portion 130b, includes, in addition to wireless data receiving means, wireless power transfer means such as an RF coil which inductively couples to an implanted coil, signal processing circuitry, an embedded power supply such as a battery, and data transfer means. The data transfer means of processing unit second portion 130b may be wired or wireless, and transfer data to one or more of: implanted processing unit first portion 130a; a different implanted device; and an external device such as an additional component of the processing unit of the present invention, a controlled device of the present invention or a computer device such as a configuration computer with Internet access, all not shown.

Referring back to FIG. 4, electrodes 212 transfer the detected cellular signals to processing unit first portion 130a via array wires 221 and wire bundle 220. Wire bundle 220 includes multiple conductive elements, and array wires 221, which preferably include a conductor for each electrode of sensor 200. Also included in wire bundle 220 are two conductors, first reference wire 222 and second reference wire 223 each of which is placed in an area in relative proximity to sensor 200 such as on the surface of brain 250 near the insertion location. First reference wire 222 and second reference wire 223 may be redundant, and provide reference signals used by one or more signal processing elements of the processing unit of the present invention to process the cellular signal data detected by one or more electrodes. In an alternative embodiment, not shown, sensor 200 consists of multiple discrete components and multiple bundles of wires connect to one or more discrete components of the processing unit, such as processing unit first portion 130a. In another alternative embodiment, not shown, cellular signals detected by sensor 200 are transmitted to processing unit 130a via wireless technologies, such as infrared communication incorporated into an electronic module of sensor 200, such transmissions penetrating the skull of the patient, and obviating the need for wire bundle 220, array wires 221 and any physical conduit passing through skull 260 after the surgical implantation procedure is completed.

Processing unit first portion 130a and processing unit second portion 130b independently or in combination preprocess the received cellular signals (e.g., impedance matching, noise filtering, or amplifying), digitize them, and further process the cellular signals to extract neural data that processing unit second portion 130b may then transmit to an external device (not shown), such as an additional processing unit component and/or any device to be controlled by the processed multicellular signals. For example, the external device may decode the received neural data into control signals for controlling a prosthetic limb or limb assist device or for controlling a computer cursor. In an alternative embodiment, the external device may analyze the neural data for a variety of other purposes. In another alternative embodiment, the device receiving transmissions from processing unit second portion 130b is an implanted device. Processing unit first portion 130a and processing unit second portion 130b independently or in combination include signal processing circuitry to perform multiple signal processing functions including but not limited to: amplification, filtering, sorting, conditioning, translating, interpreting, encoding, decoding, combining, extracting, sampling, multiplexing, analog to digital converting, digital to analog converting, mathematically transforming and/or otherwise processing cellular signals to generate a control signal for transmission to a controlled device. Processing unit first portion 130a and processing unit second portion 130b may include one or more components to assist in processing the multicellular signals or to perform additional functions. These components include but are not limited to: a temperature sensor; a pressure sensor; a strain gauge; an accelerometer; a volume sensor; an electrode; an array of electrodes; an audio transducer; a mechanical vibrator; a drug delivery device; a magnetic field generator; a photo detector element; a camera or other visualization apparatus; a wireless communication element; a light producing element; an electrical stimulator; a physiologic sensor; a heating element and a cooling element.

Processing unit first portion 130a transmits raw or processed cellular signal data to processing unit second portion 130b through integrated wireless communication means, such as the infrared communication means of FIG. 4, or alternative means including but not limited to radiofrequency communications, other optical communications, inductive communications, ultrasound communications and microwave communications. In a preferred, alternate embodiment, processing unit first portion 130a includes both infrared communication means for short-range high baud rate communication, and radiofrequency communication means for longer range, lower baud rate communication. This wireless transfer allows sensor 200 and processing unit first portion 130a to be completely implanted under the skin of the patient, avoiding the need for implanted devices that require protrusion of a portion of the device or wired connections through the skin surface. In an alternative embodiment, a through the skin pedestal connector is utilized between either the implanted sensor 200 or processing unit first portion 130a and an external component. Processing unit first portion 130a includes a coil, not shown, which receives power through inductive coupling, on a continual or intermittent basis from an external power transmitting device such as processing unit second portion 130b. The inductive coupling power transfer configuration obviates the need for any permanent power supply, such as a battery, integral to processing unit first portion 130a.

In addition to or in place of power transmission, the integrated coil of processing unit first portion 130a and its associated circuitry may receive data from an external coil whose signal is modulated in correlation to a specific data signal. The power and data can be delivered to processing unit first portion 130a simultaneously such as through simple modulation schemes in the power transfer that are decoded into data for processing unit first portion 130a to use, store or facilitate another function. A second data transfer means, in addition to a wireless means such as an infrared LED, can be accomplished by modulating a signal in the coil of processing unit first portion 130a that data is transmitted from the implant to an external device including a coil and decoding elements. In a preferred embodiment, the processing unit first portion 130a included an embedded ID, which can be wirelessly transmitted to the processing unit second portion 130b or a separate discrete component via the various wireless transmission means described above. In another preferred embodiment, processing unit second portion 130b includes means of confirming proper ID from processing unit first portion 130a and processing unit second portion 130b also included an embedded ID.

Processing unit first portion 130a and processing unit second portion 130b may independently or in combination also conduct adaptive processing of the received cellular signals by changing one or more parameters of the system to achieve acceptable or improved performance. Examples of adaptive processing include, but are not limited to, changing a system configuration parameter during a system configuration, changing a method of encoding neural or other cellular signal data, changing the type, subset, or amount of cellular signal data that is processed, or changing a method of decoding neural or other cellular signal data. Changing an encoding method may include changing neuron spike sorting methodology, calculations, thresholds, or pattern recognition methodologies. Changing a decoding methodology may include changing variables, coefficients, algorithms, and/or filter selections. Other examples of adaptive processing may include changing over time the type or combination of types of signals processed, such as EEG, ECOG, LFP, neural spikes, or other cellular signal types.

Processing unit first portion 130a and processing unit second portion 130b may independently or in combination also transmit electrical signals to one or more electrodes 212 such as to stimulate, polarize, hyperpolarize or otherwise cause an effect on one or more cells of neighboring tissue. Specific electrodes may record cellular signals only, or deliver energy only, and specific electrodes may provide both functions. In an alternative embodiment, a separate device, not shown but preferably an implanted device with the ability to independently or in combination provide an electrical signal to multiple electrodes, delivers stimulating energy to one or more electrodes 212 or different electrodes, also not shown. Stimulating electrodes in various locations can transmit signals to the central nervous system, peripheral nervous system, other body systems, body organs, muscles and other tissue or cells. The transmission of these signals is used to perform one or more functions including but not limited to: pain therapy; muscle stimulation; seizure disruption; stroke rehabilitation; coma recovery; and patient feedback.

In an alternative embodiment, not shown, processing unit first portion 130a, and potentially additional signal processing functions are integrated into sensor 200, such as through the use of a bonded electronic microchip. In another alternative embodiment, processing unit first portion 130a may also receive non-neural cellular signals and/or other biologic signals, such as from an implanted sensor. These signals may be in addition to received neural multicellular signals, and they may include but are not limited to: EKG signals, respiration signals, blood pressure signals, electromyographic activity signals and glucose level signals. Such biological signals may be used to change the state of the biological interface system of the present invention, or one of its discrete components. Such state changes include but are not limited to: turn system or component on or off; to begin a configuration routine; to initiate or conclude a step of a configuration or other routine; and to start or stop another system function. In another alternative embodiment, processing unit first portion 130a and processing unit second portion 130b independently or in combination produce one or more additional processed signals, to additionally control the controlled device of the present invention or to control one or more additional controlled devices.

In an alternative, preferred configuration of implanted components, not shown, a discrete component such as a sensor of the present invention is implanted within the cranium of the patient, such as sensor 200 of FIG. 4, a processing unit or a portion of a processing unit of the present invention is implanted in the torso of the patient, and one or more discrete components are external to the body of the patient. The processing unit may receive multicellular signals from the sensor via wired, including conductive wires and optic fibers, or wireless communication. The sensor 200 preferably includes signal processing means including signal processing up to and including digitizing the multicellular signals. In another alternative embodiment, preferably an acute (less than 24 hours) or sub-chronic (less than 30 days) application, a through the skin, or transcutaneous device is used to transmit or enable the transmission of the multicellular signals, and/or a derivative or pre-processed form of the multicellular signals.

Referring now to FIG. 5, a biological interface system 100 is shown consisting of implanted components, not shown, and components external to the body of a patient 500. A sensor for detecting multicellular signals, not shown and preferably a two dimensional array of multiple protruding electrodes, has been implanted in the brain of patient 500, in an area such as the motor cortex. In a preferred embodiment, the sensor is placed in an area to record multicellular signals that are under voluntary control of the patient. Alternatively or additionally to the two dimensional array, the sensor may include one or more wires or wire bundles which include a plurality of electrodes. Patient 500 of FIG. 5 is shown as a human being, but other mammals and life forms that produce recordable multicellular signals would also be applicable. Patient 500 may be a patient with a spinal cord injury or afflicted with a neurological disease that has resulted in a loss of voluntary control of various muscles within the patient's body. Alternatively or additionally, patient 500 may have lost a limb, and system 100 will include a prosthetic limb as its controlled device. Numerous types of patients, including healthy individuals, are applicable to the system of the present invention. The patient of the present invention may be a quadriplegic, a paraplegic, an amputee, a spinal cord injury victim or an otherwise physically impaired person. Alternatively or in addition, Patient 500 may have been diagnosed with one or more of: obesity, an eating disorder, a neurological disorder, a psychiatric disorder, a cardiovascular disorder, an endocrine disorder, sexual dysfunction, incontinence, a hearing disorder, a visual disorder, sleeping disorder, a movement disorder, a speech disorder, physical injury, migraine headaches or chronic pain. System 100 can be used to treat one or more medical conditions of patient 500, or to restore, partially restore, replace or partially replace a lost function of patient 500.

Alternatively, system 100 can be utilized by patient 500 to enhance performance, such as if patient 500 did not have a disease or condition from which a therapy or restorative device could provide benefit, but did have an occupation wherein thought control of a device provided an otherwise unachieved advancement in healthcare, crisis management and national defense. Thought control of a device can be advantageous in numerous healthy individuals including but not limited to: a surgeon, such as an individual surgeon using thought control to maneuver three or more robotic arms in a complex laparoscopic procedure or a surgeon controlling various instruments at a location remote from the instruments and the surgical procedure; a crisis control expert, such as a person who in attempting to minimize death and injury uses thought control to communicate different pieces of information and/or control multiple pieces of equipment, such as urban search and rescue equipment, simultaneously during an event such as an earthquake or other disaster, both natural disasters and those caused by man; a member of a bomb squad, such as an expert who uses thoughts to control multiple robots and/or robotic arms to remotely diffuse a bomb; and military personnel who use thought control to communicate with personnel and control multiple pieces of defense equipment, such as artillery, aircraft, watercraft, land vehicles and reconnaissance robots. It should be noted that the above advantages of system 100 to a healthy individual are also advantages achieved in a patient such as a quadriplegic or paraplegic. In other words, a quadriplegic could provide significant benefit to society, such as in controlling multiple bomb diffusing robots, in addition to his or her own ambulation and other quality of life devices. Patients undergoing implantation and use of the system 100 of the present invention may provide numerous occupational and other functions not available to individuals that do not have the biological interface system of the present invention.

The sensor electrodes of system 100 can be used to detect various multicellular signals as has been described in detail in reference to FIG. 4 hereabove. The sensor is connected via a multi-conductor cable, not shown but also implanted in patient 500, to an implanted portion of the processing unit which includes some signal processing elements as well as wireless communication means as has been described in detail in reference to FIG. 4. The implanted multi-conductor cable preferably includes a separate conductor for each electrode, as well as additional conductors to serve other purposes, such as providing reference signals and ground. A second portion of the processing unit, processing unit second portion 130b receives the wireless communications from the implanted portion. Processing unit second portion 130b is removably located just above the ear of patient 500, such as to be aligned with an infrared data transmission element of the implanted device. Multicellular signals or derivatives of the multicellular signals are transmitted from the implanted processing unit component to processing unit second portion 130b for further processing. The processing unit components of system 100 perform various signal processing functions as have been described in detail in reference to FIG. 4. The processing unit may process signals that are mathematically combined, such as the combining of neuron spikes that are first separated using spike discrimination methods, these methods known to those of skill in the art. In alternative embodiments, the processing unit may consist of multiple components or a single component; each of the processing unit components can be fully implanted in patient 500, be external to the body, or be implanted with a portion of the component exiting through the skin.

In FIG. 5, a first controlled device is a computer, CPU 305 that is attached to monitor 302 and integrated into configuration cart 121. Through the use of system 100, patient 500 can control one or more computer functions including but not limited to: an on/off function, a reset function, a language function, a modem function, a printer function, an Internet function, a cursor, a keyboard, a joystick, a trackball or other input device. Each function may be controlled individually or in combination. System 100 includes a second controlled device, wheelchair 310. Numerous other controlled devices can be included in the systems of this application, individually or in combination, including but not limited to: a computer; a computer display; a mouse; a cursor; a joystick; a personal data assistant; a robot or robotic component; a computer controlled device; a teleoperated device; a communication device or system; a vehicle such as a wheelchair; an adjustable bed; an adjustable chair; a remote controlled device; a Functional Electrical Stimulator device or system; a muscle stimulator; an exoskeletal robot brace; an artificial or prosthetic limb; a vision enhancing device; a vision restoring device; a hearing enhancing device; a hearing restoring device; a movement assist device; medical therapeutic equipment such as a drug delivery apparatus; medical diagnostic equipment such as epilepsy monitoring apparatus; other medical equipment such as a bladder control device, a bowel control device and a human enhancement device; closed loop medical equipment and other controllable devices applicable to patients with some form of paralysis or diminished function as well as any device that may be utilized under direct brain or thought control in either a healthy or unhealthy patient.

Processing unit second portion 130b includes a unique electronic ID, such as a unique serial number or any alphanumeric or other retrievable, identifiable code associated uniquely with the system 100 of patient 500. The unique electronic identifier may take many different forms in processing unit second portion 130b, such as a piece of electronic data stored in a memory module; a semiconductor element or chip that can be read electronically via serial, parallel or telemetric communication; pins or other conductive parts that can be shorted or otherwise connected to each other or to a controlled impedance, voltage or ground, to create a unique code; pins or other parts that can be masked to create a binary or serial code; combinations of different impedances used to create a serial code that can be read or measured from contacts, features that can be optically scanned and read by patterns and/or colors; mechanical patterns that can be read by mechanical or electrical detection means or by mechanical fit, a radio frequency ID or other frequency spectral codes sensed by radiofrequency or electromagnetic fields, pads and/or other marking features that may be masked to be included or excluded to represent a serial code, or any other digital or analog code that can be retrieved from the discrete component.

Alternatively or in addition to embedding the unique electronic ID in processing unit second portion 130b, the unique electronic ID can be embedded in one or more implanted discrete components. Under certain circumstances, processing unit second portion 130b or another external or implanted component may need to be replaced, temporarily or permanently. Under these circumstances, a system compatibility check between the new component and the remaining system components can be confirmed at the time of the repair or replacement surgery through the use of the embedded unique electronic ID. The unique electronic ID can be embedded in one or more of the discrete components at the time of manufacture, or at a later date such as at the time of any clinical procedure involving the system, such as a surgery to implant the sensor electrodes into the brain of patient 500. Alternatively, the unique electronic ID may be embedded in one or more of the discrete components at an even later date such as during a system configuration routine such as a calibration routine.

Referring again to FIG. 5, processing unit second portion 130b communicates with one or more discrete components of system 100 via wireless communication means. Processing unit second portion 130b communicates with selector module 400, a component utilized to select the specific device or devices to be controlled by the processed signals of system 100. Selector module 400 includes a touch screen set of buttons, input element 402, used to perform the selection process. Processing unit second portion 130b also communicates with controlled device CPU 305, such as to control a cursor, joystick, keyboard or other function of CPU 305. Processing unit second portion 130b further communicates with processing unit third portion 130c. Processing unit third portion 130c provides additional signal processing functions, as have been described above, to control wheelchair 310. An additional processing unit discrete component, processing unit fourth portion 130d, is included to perform additional processing of the multicellular signals and/or derivatives of these processed signals and/or processing of additional information, such collective processing used to control one or more additional controlled devices of the present invention, not shown. System 100 of FIG. 5 utilizes selector module 400 to select one or more of CPU 305, wheelchair 310 or another controlled device to be controlled by the processed signals produced by the processing unit of the present invention. In system 100 of FIG. 5, one set of processed signals emanate from one portion of the processing unit, processing unit second portion 130b, and a different set of processed signals emanate from a different portion of the processing unit, processing unit third portion 130c.

The various components of system 100 communicate with wireless transmission means, however it should be appreciated that physical cables can be used to transfer data alternatively or in addition to wireless means. These physical cables may include electrical wires, optical fibers, sound wave guide conduits, and other physical means of transmitting data and/or power and any combination of those means.

Referring back to FIG. 5, a qualified individual, operator 110 in cooperation with patient 500, is performing a patient training routine, one of numerous configuration programs or routines of the system. In an alternative embodiment, patient 500 is the operator of the patient training routine or other configuration routine. The patient training routine is shown being performed with controlled device 305. Displayed on monitor 302 is planned trajectory 711, system controlled target 712 and patient controlled object 713. In the performance of the patient training routine, multiple time varying stimulus, such as a moving system controlled target 712 are provided to the patient such that the patient can imagine moving that target, and a set of multicellular signal data can be collected by the processing unit to produce one or more algorithms to produce the processed signals of the present invention. In a preferred embodiment, after a first set of multicellular signal data is collected, and a first transfer function for producing processed signals is developed, a second set of time varying stimulus is provided in combination with a patient controlled object, such as patient controlled object 713. During the time that the patient tries to mimic the motion of the system controlled target 712 with the visual feedback of the patient controlled target 713, and a second set of multicellular signal data is collected and a second, improved transfer function is produced by the system. Additional forms of feedback can be provided to the patient, such as tactile transducer 701 shown attached to patient 500's neck, and speaker 702 shown attached to processing unit third portion 130c fixedly mounted to the back of controlled wheelchair 310. Speaker 702 and tactile transducer 701 can provide feedback in the form of a time varying stimulus, a derivative of the multicellular signals, and/or a representation of the processed signals as controlled by patient 500

In a preferred embodiment, one or more system configuration routines can be performed without an operator, with the patient as the operator, or with an operator at a remote location such as when the system of the present invention is electronically connected with a computer or computer network such as the Internet. In another preferred embodiment, the patient training routine must be performed at least one time during the use of the system, preferably before patient 500 is given, by the system, full control of one or more controlled devices. For example, limited control of CPU 305 may include the ability to send and receive email but not the ability to adjust a computer-controlled thermostat. Limited control of wheelchair 310 may be to turn left or right, but not move forward or back, or to only allow travel at a limited velocity. For the purposes of this specification, limited control may also include no control of one or more controlled devices. Each controlled device will have different parameters limited by system 100 when patient 500 has not been given full control. In a preferred embodiment, the selection of these parameters; the values to be limited; the criteria for achieving full control such as the value of a success threshold achieved during a system configuration routine such as a patient training routine; and combinations of these, are modified only in a secured way such as only by a clinician utilizing electronic or mechanical keys or passwords.

In addition to successful completion of the patient training routine, completion of one or more other configuration routines may be required for patient 500 to have full control of one or more controlled devices, or multiple successful completions of a single routine. Success is preferably measured through the measurement of one or more performance parameters during or after the configuration routine. Success will be achieved by a performance parameter being above a threshold value, such as a threshold adjustable only by a clinician, such as a clinician at a remote site utilizing a password, a user identification, an electronic ID and/or a mechanical key. These configuration routines are utilized by the system to not only determine the applicability of full control to the patient, but to set or reset one or more system configuration parameters. System configuration parameters include but are not limited to: selection of cellular signals for processing by the processing unit; criteria for the selection of cells for processing; a coefficient of a signal processing function such as amplification, filtering, sorting, conditioning, translating, interpreting, encoding, decoding, combining, extracting, sampling, multiplexing, analog to digital converting, digital to analog converting, mathematically transforming; a control signal transfer function parameter such as a transfer function coefficient, algorithm, methodology, mathematical equation, a calibration parameter such as calibration frequency; a controlled device parameter such as a controlled device boundary limit; acceptable frequency range of cellular activity; selection of electrodes to include; selection of cellular signals to include; type of frequency analysis such as power spectral density; instruction information to patient such as imagined movement type or other imagined movement instruction; type, mode or configuration of feedback during provision of processed signals to patient; calibration parameter such as calibration duration and calibration frequency; controlled device parameter such as controlled device mode; alarm or alert threshold; and a success threshold.

As depicted in FIG. 5, operator 110 utilizes configuration apparatus 120 which includes two monitors, first configuration monitor 122a and second configuration monitor 122b, configuration keyboard 123, and configuration CPU 125, to perform a calibration routine or other system configuration process such as a patient training routine, algorithm and algorithm parameter selection and output device setup. The configuration routines, such as the patient training routine, include software programs and hardware required to perform the configuration. The embedded software and/or hardware may be included in the processing unit, such as processing unit second portion 130b, be included in selector module 400, be incorporated into configuration apparatus 120, a controlled device, or combinations of these. Configuration apparatus 120 may include additional input devices, such as a mouse or joystick, or an input device for a patient with limited motion, such as a tongue switch; a tongue palate switch; a chin joystick; a Sip n' Puff joystick controller; an eye tracker device; a head tracker device; an EMG switch such as an eyebrow EMG switch; an EEG activated switch; and a speech recognition device, all not shown.

Configuration apparatus 120 may include various elements, functions and data including but not limited to: memory storage for future recall of configuration activities, operator qualification routines, standard human data, standard synthesized or artificial data, neuron spike discrimination software, operator security and access control, controlled device data, wireless communication means, remote (such as via the Internet) configuration communication means and other elements, functions and data used to provide an effective and efficient configuration on a broad base of applicable patients and a broad base of applicable controlled devices. A system electronic ID can be embedded in one or more of the discrete components at the time, including an ID embedded at the time of system configuration. In an alternative embodiment, all or part of the functionality of configuration apparatus 120 is integrated into selector module 400 such that system 100 can perform one or more configuration processes such as a calibration procedure or patient training routine, utilizing selector module 400 without the availability of configuration apparatus 120.

In order to change a system configuration parameter, system 100 includes a permission routine, such as an embedded software routine or software driven interface that allows the operator to view information and enter data into one or more components of system 100. The data entered must signify an approval of the parameter modification in order for the modification to take place. Alternatively, the permission routine may be partially or fully located in a separate device such as configuration apparatus 120 of FIG. 5, or a remote computer such as a computer that accesses system 100 via the Internet or utilizing wireless technologies. In order to access the permission routine, and/or approve the modification of the system configuration parameters, a password or security key, mechanical, electrical, electromechanical or software based, may be required of the operator. Multiple operators may be needed or required to approve a parameter modification. Each specific operator or operator type may be limited by system 100, via passwords and other control configurations, to approve the modification of only a portion of the total set of modifiable parameters of the system. Additionally or alternatively, a specific operator or operator type may be limited to only approve a modification to a parameter within a specific range of values, such as a range of values set by a clinician when the operator is a family member. Operator or operator types, hereinafter operator, include but are not limited to: a clinician, primary care clinician, surgeon, hospital technician, system 100 supplier or manufacturer technician, computer technician, family member, immediate family member, caregiver and patient.

In a preferred embodiment, the system 100 of FIG. 5 includes an interrogation function, which interrogates the system to retrieve certain information such as on the demand of an operator. Based on the analysis of the information, a recommendation for a parameter value change can be made available to the operator, such as by automatic configuration or calibration routines that are initiated by the operator initiated interrogation function. After viewing the modification, the appropriate operator would approve the change via the permission routine, such as using a computer mouse to click “OK” on a confirmation box displayed on a display monitor, or a more sophisticated, password controlled methodology.

In a preferred embodiment, an automatic or semi-automatic configuration function or routine is embedded in system 100. This embedded configuration routine can be used in place of a configuration routine performed manually by Operator 110 as is described hereabove, or can be used in conjunction with one or more manual configurations. Automatic and/or semi-automatic configuration triggering event or causes can take many forms including but not limited to: monitoring of cellular activity, wherein the system automatically changes which particular signals are chosen to produce the processed signals; running parallel algorithms in the background of the one or more algorithms currently used to create the processed signals, and changing one or more algorithms when improved performance is identified in the background event; monitoring of one or more system functions, such as alarm or warning condition events or frequency of events, wherein the automated system shuts down one or more functions and/or improves performance by changing a relevant variable; and other methods that monitor one or more pieces of system data, identify an issue or potential improvement, and determine new parameters that would reduce the issue or achieve an improvement. In a preferred embodiment of the disclosed invention, when specific system configuration parameters are identified, by an automated or semi-automated calibration or other configuration routine, to be modified for the reasons described above, an integral permission routine of the system requires approval of a specific operator when one or more of the system configuration parameters are modified.

Operator 110 may be a clinician, technician, caregiver, patient family member or even the patient themselves in some circumstances. Multiple operators may be needed or required to perform a configuration routine or approve a modification of a system configuration parameter, and each operator may be limited by system 100, via passwords and other control configurations, to only perform or access specific functions. For example, only the clinician may be able to change specific critical parameters, or set upper and lower limits on other parameters, while a caregiver, or the patient, may not be able to access those portions of the configuration procedure or the permission procedure. The configuration routine includes the setting of numerous parameters needed by system 100 to properly control one or more controlled devices. The parameters include but are not limited to various signal conditioning parameters as well as selection and de-selection of specific multicellular signals for processing to generate the device control creating a subset of signals received from the sensor to be processed. The various signal conditioning parameters include, but are not limited to, threshold levels for amplitude sorting, other sorting and pattern recognition parameters, amplification parameters, filter parameters, signal conditioning parameters, signal translating parameters, signal interpreting parameters, signal encoding and decoding parameters, signal combining parameters, signal extracting parameters, mathematical parameters including transformation coefficients and other signal processing parameters used to generate a control signal for transmission to a controlled device.

The configuration routine will result in the setting of various system configuration output parameters, all such parameters to be considered system configuration parameters of the system of the present invention. Configuration output parameters may consist of but are not limited to: electrode selection, cellular signal selection, neuron spike selection, electrocorticogram signal selection, local field potential signal selection, electroencephalogram signal selection, sampling rate by signal, sampling rate by group of signals, amplification by signal, amplification by group of signals, filter parameters by signal and filter parameters by group of signals. In a preferred embodiment, the configuration output parameters are stored in memory in one or more discrete components, and the parameters are linked to the system's unique electronic ID.

Calibration, patient training, and other configuration routines, including manual, automatic and semi-automatic routines, may be performed on a periodic basis, and may include the selection and deselection of specific cellular signals over time. The initial configuration routine may include initial values, or starting points, for one or more of the configuration output parameters. Setting initial values of specific parameters, may invoke a permission routine. Subsequent configuration routines may involve utilizing previous configuration output parameters that have been stored in a memory storage element of system 100. Subsequent configuration routines may be shorter in duration than an initial configuration and may require less patient involvement. Subsequent configuration routine results may be compared to previous configuration results, and system 100 may require a repeat of configuration if certain comparative performance is not achieved.

The configuration routine may include the steps of (a) setting a preliminary set of configuration output parameters; (b) generating processed signals to control the controlled device; (c) measuring the performance of the controlled device control; and (d) modifying the configuration output parameters. The configuration routine may further include the steps of repeating steps (b) through (d). The configuration routine may also require invoking a permission routine.

In the performance of a configuration routine, the operator 110 may involve patient 500 or perform steps that do not involve the patient. In the patient training routine and other routines, the operator 110 may have patient 500 imagine one or more particular movements, imagined states, or other imagined events, such as a memory, an emotion, the thought of being hot or cold, or other imagined event not necessarily associated with movement. The patient participation may include the patient training routine providing one or more time varying stimulus, such as audio cues, visual cues, olfactory cues, gustatory cues, tactile cues, moving objects on a display such as a computer screen, moving mechanical devices such as a robotic arm or a prosthetic limb, moving a part of the patient's body such as with an exoskeleton or FES implant, changing audio signals, changing electrical stimulation such as cortical stimulation, moving a vehicle such as a wheelchair or car; moving a model of a vehicle; moving a transportation device; and other sensory stimulus. The imagined movements may include the imagined movement of a part of the body, such as a limb, arm, wrist, finger, shoulder, neck, leg, angle, and toe, as well as imagining moving to a location, moving in a direction, moving at a velocity or acceleration.

Referring back to FIG. 5, the patient imagines moving system controlled target 712 along planned trajectory 711, as target 712 is moving as controlled by the system or manually by an operator. The current processed signal, hereinafter a representation of the processed signal, available by applying a transfer function to the multicellular signals detected during the imagined movement or other step of the patient training routine, is displayed in the form of control of patient controlled target 713. The transfer function is preferably based on multicellular signals stored during a previous imagined movement, or multiple previous imagined movements, preferably two or more sets of states of time varying stimulus. The representation of the processed signals may mimic the time varying stimulus, similar to patient controlled object 713 being a similar form to system controlled object 712. Alternatively, the time varying stimulus and representation of the processed signals may take different forms, such as a time varying stimulus consisting of an object on a visual display, wherein the representation is a moving mechanical structure, or the stimulus being a moving mechanical structure and the representation consisting of an object on a visual display. The representation of the processed signals can be provided to the patient in visual form such as a visual representation of limb motion displayed on a computer monitor, or in one or more sensory forms such as auditory, olfactory, gustatory, and electrical stimulation such as cortical stimulation. The representation of the processed signals can be provided in combinations of these and other forms.

In a preferred embodiment, the first patient training step does not include patient controlled object 713 or it includes a patient controlled target whose processed signals are not based on a set of multicellular signals collected during a previous imagined movement. Multiple steps of providing a set of states of the time varying stimulus and recording the multicellular signal data may involve different subsets of cells from which the multicellular signals are detected. Also, different sets of states of time varying stimulus may have different numbers of cells in each. Alternative to the system controlled target 712 along planned trajectory 711, the patient may imagine movements while viewing a time varying stimulus comprising a video or animation of a person performing the specific movement pattern. In a preferred embodiment, this visual feedback is shown from the patient's perspective, such as a video taken from the person performing the motion's own eye level and directional view. Multiple motion patterns and multiple corresponding videos may be available to improve or otherwise enhance the patient training process. The patient training routine temporally correlates a set of states of the time varying stimulus with the set of multicellular signal signals captured and stored during that time period, such that a transfer function can be developed for future training or controlled device control. Correlations can be based on numerous variables of the motion including but not limited to: position, velocity and acceleration of the time varying stimulus; a patient physiologic parameter such as heart rate; a controlled device parameter; a system environment parameter; a password controlled parameter; a clinician controlled parameter; and a patient training routine parameter. In the patient training routine of FIG. 5, the controlled device, CPU 305 and controlled monitor 302 are used in the patient training routine to display the time varying stimulus as well as the representation of the processed signal. In a subsequent step, wheelchair 310 can also be employed, such as by a system controlling the wheelchair while the patient imagines the control, the wheelchair movement being the time varying stimulus.

During the time period that a set of states of the time varying stimulus is applied, multicellular signal data detected by the implanted sensor is stored and temporally correlated to that set of states of the time varying stimulus provided to the patient. In a preferred embodiment, the system of the present invention includes a second patient training routine and a second controlled device, wherein the first patient training routine is used to configure the first controlled device and the second patient training routine is used to configure the second controlled device. The two patient training routines may include different time varying stimulus, chosen to optimize the routine for the specific controlled device, such as a moving cursor for a computer mouse control system, and a computer simulated prosthetic limb for a prosthetic limb control system. In a preferred system, the first controlled device is a prosthetic arm and the second controlled device is a prosthetic leg, this system having two different time varying stimulus in the two corresponding patient training routines. In another preferred system, the first controlled device is a prosthetic arm and the second controlled device is a wheelchair, this system also having two different time varying stimulus in the two corresponding patient routines. In an alternative, preferred embodiment, a controlled device surrogate is utilized in the patient training routine. The controlled device surrogate preferably has a larger value of one or more of: degrees of freedom; resolution; modes; discrete states; functions; and boundary conditions. Numerous boundary conditions with greater values for the surrogate device can be employed, such boundary conditions as: maximum distance; maximum velocity; maximum acceleration; maximum force; maximum torque; rotation; and position. The surrogate device employing larger values of these parameters creates the scenario wherein the patient is trained and/or tested with a device of more complexity than the eventual controlled device to be used.

The time varying stimulus may be supplied to the patient in numerous forms such as visual, tactile, olfactory, gustatory, and electrical stimulation such as cortical stimulation. The time varying stimulus may be moved around manually, automatically produced and controlled by a component of the system such as the processing unit, or produced by a separate device. The time varying stimulus may include continuous or semi-continuous motion of an object, such as an object moving on a visual display, a mechanical object moving in space, or a part of the patient's body moving in space. The time varying stimulus may be of a short duration, such as an object appearing and disappearing quickly on a display, or a flash of light.

In a preferred embodiment, the patient training routine includes multiple forms of feedback, in addition to the time varying stimulus, such feedback provided to the patient in one or more forms including but not limited to: visual; tactile; auditory; olfactory; gustatory; and electrical stimulation. The additional feedback may be a derivative of the multicellular signals, such as visual or audio feedback of one or more neuron spike modulation rates. Different forms of feedback may be provided as based on a particular device to be controlled by the processed signals. Numerous parameters for the time varying stimulus and other feedback may be adjustable, such as by the operator or patient, these parameters including but not limited to: sound volume and frequency; display brightness, contrast, size and resolution; display object size; electrical current parameter such as current or voltage; mechanical or visual object size, color, configuration, velocity or acceleration; and combinations of these.

A configuration routine such as a calibration or patient training routine will utilize one or more configuration input parameters to determine one or more system output parameters used to develop a processed signal transfer function. In addition to the multicellular signals themselves, system or controlled device performance criteria can be utilized. Other configuration input parameters include various properties associated with the multicellular signals including one or more of: signal to noise ratio, frequency of signal, amplitude of signal, neuron firing rate, average neuron firing rate, standard deviation in neuron firing rate, modulation of neuron firing rate as well as a mathematical analysis of any signal property including but not limited to modulation of any signal property. Additional configuration input parameters include but are not limited to: system performance criteria, controlled device electrical time constants, controlled device mechanical time constants, other controlled device criteria, types of electrodes, number of electrodes, patient activity during configuration, target number of signals required, patient disease state, patient condition, patient age and other patient parameters and event based (such as a patient imagined movement event) variations in signal properties including neuron firing rate activity. In a preferred embodiment, one or more configuration input parameters are stored in memory and linked to the embedded, specific, unique electronic identifier. All configuration input parameters shall be considered a system configuration parameter of the system of the present invention.

It may be desirous for the configuration routine to exclude one or more multicellular signals based on a desire to avoid signals that respond to certain patient active functions, such as non-paralyzed functions, or even certain imagined states. The configuration routine may include having the patient imagine a particular movement or state, and based on sufficient signal activity such as firing rate or modulation of firing rate, exclude that signal from the signal processing based on that particular undesired imagined movement or imagined state. Alternatively real movement accomplished by the patient may also be utilized to exclude certain multicellular signals emanating from specific electrodes of the sensor. In a preferred embodiment, an automated or semi-automated calibration or other configuration routine may include through addition, or exclude through deletion, a signal based on insufficient activity during known patient movements.

The configuration routines of the system of the present invention, such as a patient training routine in which a time varying stimulus is provided to the patient, may conduct adaptive processing, such as adapting between uses or within a single patient training routine. The adaptation may be caused by a superior or inadequate level of performance, as compared to a threshold value, such as an adjustable threshold. In a preferred embodiment, performance during a patient training routine above a threshold value causes the duration of the routine to decrease, and performance below a threshold value causes the duration of the routine to increase. Control of the controlled device or surrogate controlled device is a preferred way of measuring performance. Alternatively, a change in multicellular signals, such as a change in modulation rate may cause an adaptation to occur. A monitored difference is a first patient training event and a second patient training event, such as a difference in signal modulation, may cause an adaptation in the patient training routine, such as to preferentially choose one time varying stimulus over another time varying stimulus. Other causes include a change to a patient parameter, such as the level of patience consciousness. In a preferred embodiment, at a low level of consciousness, the patient training routine changes or discontinues. The level of consciousness may be determined by the multicellular signals detected by the sensor. Alternatively, the level of consciousness can be detected utilizing a separate sensor, such as a sensor to detect EEG or LFP signals. The patient training routine may automatically adapt, such as due to a calculation performed by the processing unit, or may adapt due to operator input.

The systems of the present invention, such as system 100 of FIG. 5, include a processing unit that processes multicellular signals received from patient 500. Processing unit second portion 130b and other processing unit components, singly or in combination, perform one or more functions. The functions performed by the processing unit include but are not limited to: producing the processed signals; transferring data to a separate device; receiving data from a separate device; producing processed signals for a second controlled device; activating an alarm, alert or warning; shutting down a part of or the entire system; ceasing control of a controlled device; storing data and performing a configuration.

In order for the processing unit of system 100 to perform one or more functions, one or more system configuration parameters are utilized. These parameters include pieces of data stored in, sent to, or received from, any component of system 100, including but not limited to: the sensor; a processing unit component; processing unit second portion 130b; or a controlled device. Parameters can be received from devices outside of system 100 as well, such as configuration apparatus 120, a separate medical therapeutic or diagnostic device, a separate Internet based device or a separate wireless device. These parameters can be numeric or alphanumeric data, and can change over time, either automatically or through an operator involved configuration or other procedure.

The processing unit, or other component of system 100 may produce multiple processed signals for controlling one or more controlled device. This second processed signals may be based on multicellular signals of the sensor, such as the same set of cells as the first processed signals are based on, or a different set of cells emanating signals. The signal processing used to produce the additional processed signals can be the same as the first, or utilize different processing, such as different transfer functions. Transfer functions may include different algorithms, coefficients such as scaling factors, different types of feedback, and other transfer function variations. Alternatively, the additional processed signals may be based on signals not received from the sensor in which the first processed signals are derived. An additional sensor, such as a similar or dissimilar sensor, may provide the signals to produce the additional processed signals, or the system may receive a signal from an included input device such as a tongue switch; tongue palate switch; chin joystick; Sip n' Puff joystick controller; eye gaze tracker; head tracker; EMG switch such as eyebrow EMG switch; EEG activated switch; speech recognition device; and combinations thereof. The additional processed signals may be derived from a monitored biological signal such as a signal based on eye motion; eyelid motion; facial muscle activation or other electromyographic activity; heart rate; EEG; LFP; respiration; and combinations thereof. In creating the additional processed signals, the processing unit may convert these alternative input signals into a digital signal, such as a digital signal used to change the state of the system, such as a change in state of an integrated configuration routine.

Referring now to FIG. 6, another preferred embodiment of the joint movement device of the present invention is illustrated, wherein the joint movement device is for applying a force to two or more joints of the patient, such as the elbow, the wrist, and joints of the hand as depicted. The joint movement device is similar to the joint movement device of FIG. 3 with the addition of a torque generating assembly for applying a torsional force to the patient's elbow. Items with the same reference numbers have the same functionality and embodiments as have been described hereabove in reference to FIG. 3. Hand and elbow apparatus 802, shown from the palm side orientation, is placed over the hand a patient 500, such that lost or compromised control of patient 500's elbow, hand and wrist can be restored. Assembly 802 is configured to open, close and partially close the fist, such as to grasp an object, as well as independently control the fingers of patient 500. In addition, assembly 801 can be used to precisely flex the wrist of patient 500. Glove 810, an elastic, flexible material such as an elastic fabric, acts as a force translating structure, glove 810 being in close contract with multiple portions of patient 500 from the elbow to the tips of one or more fingers. In a preferred embodiment, glove 810 substantially surrounds, such as making contact with more than half the skin surface area from the proximal end to the distal end of glove 810, the end of the elbow to the tip of the fingers respectively.

In addition to applying a force to the wrist and one or more finger joints, hand and elbow apparatus 802 can controllably apply a force to the elbow on the same arm of patient 500 as the controlled wrist and hand. Powered elbow joint 850 surrounds patient 500's elbow, and includes a pivoting assembly 852 which has a central rotational axis aligned with patient 500's elbow joint axis. Motor assembly 855, of similar construction to motor assembly 820 but preferably able to produce more torque, is attached to pivoting assembly 852 such that activation of motor assembly 855 can apply a force which results in a torsional force being applied to the elbow of patient 500. Motor assembly 855 is attached to electronic module 840 via wiring 854, such as to receive power and/or one or more drive signals. Motor assembly 855 may include one or more sensors such as a position encoders described in reference to motor assembly 820 of FIG. 3.

In a preferred embodiment, hand and elbow apparatus 802 is a controlled device of the biological interface apparatus of the present invention, wherein electronic module 840 receives processed signals for causing individual control cables 830 and 830′ to retract, applying force to one or more joints independently, or motor assembly 855 to apply force to patient 500's elbow joint. It should be noted that the biological interface of the present invention is unique in its ability to provide a sophisticated control signal enabling patient 500 to cause joint movement similar to normal hand, wrist and other joint control. The sensor of the biological interface apparatus, such as a sensor comprising multiple discrete components placed in multiple locations, can be placed in the portion of the brain's motor cortex associated with the joints to be controlled, and/or proximate to one or more nerves of the central or peripheral nervous system associated with the specific joints.

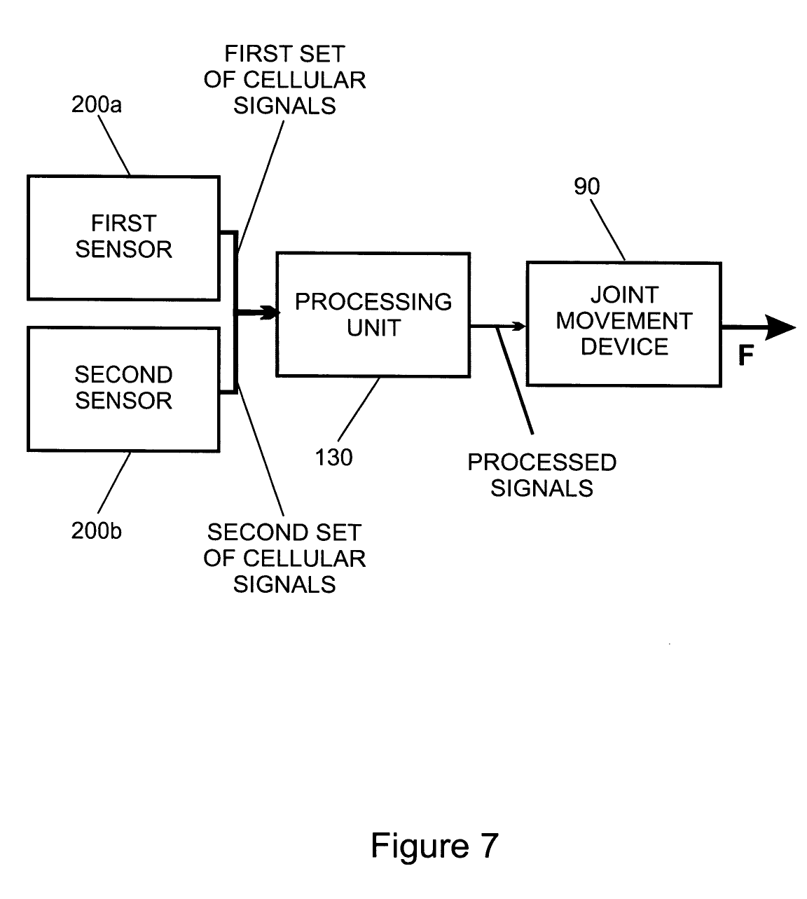

Referring now to FIG. 7, another preferred embodiment of the biological interface apparatus of the present invention is illustrated with multiple sensors placed to control a joint movement device of the apparatus. Biological interface apparatus 100 includes a first sensor 200a and a second sensor 200b, the sensors each comprising at least one electrode configured to detect a set of cellular signals emanating from one or more living cells of a patient. Processing unit 130 receives the two sets of cellular signals and processes the cellular signals to produce processed signals that are transmitted to and used to control joint movement device 90. Joint movement device 90 applies force F to one or more joints of the patient, and/or one or more joints of a prosthetic device being used by the patient.

The second sensor 200b is placed in proximity to specific cells that were previously in neurological communication with a portion of the patient limb or a portion of the patient limb replaced by a prosthetic limb. In a preferred embodiment, the joint movement device is a prosthetic limb placed over a remaining stump of the patient's arm or leg, and the second sensor 200b is placed into the most proximate nerves still emanating signals representative of patient imagined movements for the missing limb. In another preferred embodiment, the joint movement device is an exoskeleton device, such as the exoskeleton devices of FIG. 3 and FIG. 6, or an FES device, wherein the joint movement device restores function of a paralyzed or partially paralyzed limb, such as a paralysis caused by a spinal cord injury. Second sensor 200b is placed near one or more intact nerves such as nerves of the spinal cord above the injury, these nerves emanating signals representative of patient imagined movements for the paralyzed limb. Joint movement device 90 is chosen and configured as has been described in detail in reference to FIG. 1 and FIG. 2.

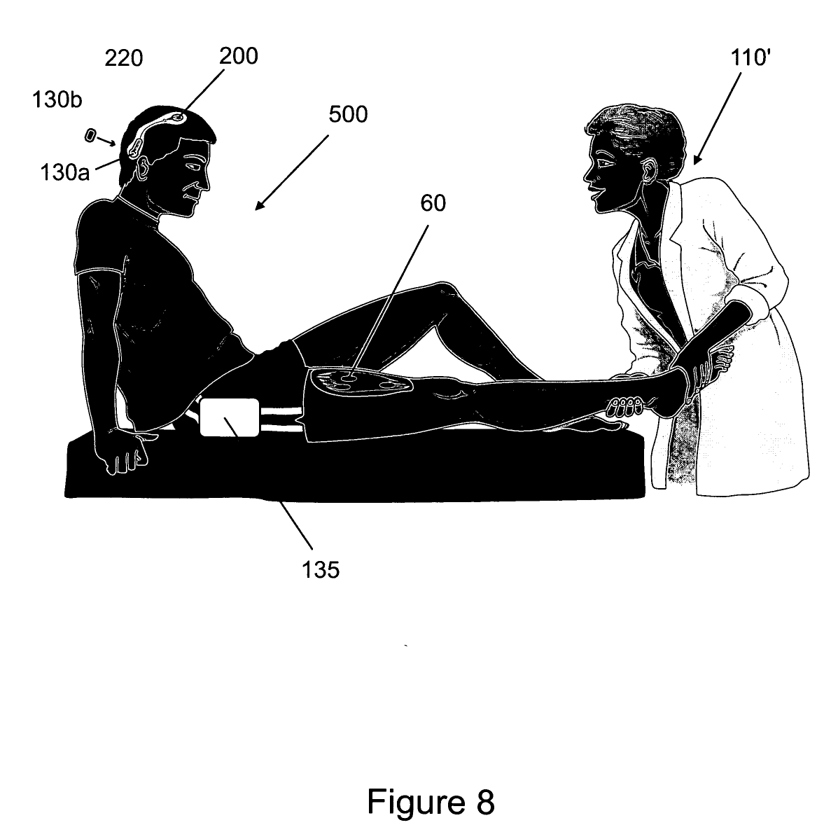

Referring now to FIG. 8, another preferred embodiment of the biological interface apparatus of the present invention is illustrated wherein a patient which an implanted joint movement device receives physical therapy. Biological interface apparatus 100, which is described in detail in reference to FIG. 4 and FIG. 5 hereabove, includes sensor 200 and a processing unit for processing the multicellular signals that are detected by sensor 200. The processing unit comprises two discrete components, processing unit first portion 130a that is implanted under the scalp of patient 500, and processing unit second portion 130b that is external to the patient. Sensor 200 is illustrated through a view of the skull that has been cutaway, sensor 200 being implanted in the motor cortex of patient 500's brain. In a preferred embodiment, sensor 200 is implanted in a portion of the motor cortex associated one or more the joints or surrogate, prosthetic joints controlled by one or more joint movement devices of apparatus 100. In a preferred embodiment, a functional MRI (fMRI) is performed prior to the surgery in which the patient imagines moving one or more target joints, and sensor 200 is located based on information output from the fMRI. A wire bundle 220 connects sensor 200 to processing unit first portion 130a, which has been placed in a recess, surgically created in patient 500's skull, viewed in FIG. 1 through a cutaway of patient 500's scalp. Wire bundle 220 includes multiple, flexible insulated wires, preferably a single wire for each electrode. In an alternative embodiment, one or more single wires carry cellular signal transmissions from two or more electrodes. The surgical procedure required for the implantation of wire bundle 220, as well as sensor 200 and processing unit first portion 130a, is described in detail in reference to FIG. 4 hereabove. Alternatively or additionally, a cellular signal sensor component may be placed in numerous locations such as the spinal cord or a peripheral nerve.

Processing unit first portion 130a transmits data, such as with RF or infrared transmission means, to a receiver of processing unit second portion 130b, which is shown as in the process of being removably placed at a location near the implant site of processing unit first portion 130a. In a preferred embodiment, magnets integral to either or both processing unit discrete components are used to maintain the components in appropriate proximity and alignment to assure accurate transmissions of data. One or more patient input devices, not shown, may be affixed to patient 500. These switches are used to provide a patient activated input signal to biological interface apparatus 100. In an alternative or additional embodiment, one or more of these switches is used to provide a patient activated input to one or more components of apparatus 100. Patient input switches incorporated into one or more apparatus, device, methods and systems of the present invention can be used in the performance of various system functions or routines and/or to initiate various system functions or routines. In a preferred embodiment, a patient input switch is used to change the state of the system, such as when the system state changes to: a reset state; the next step of a configuration routine, a stopped state; an alarm state; a message sending state, a limited control of controlled device state; and combinations thereof. Alternative to the patient input switch is a monitored biological signal that is used for a similar change of state function. Applicable monitored biological signals are selected from the group consisting of: eye motion; eyelid motion; facial muscle activation or other electromyographic activity; heart rate; EEG; LFP; respiration; and combinations thereof.

Patient 500 is a patient with limited motor function such as a paraplegic or quadriplegic. Patient 500 may be an ALS patient whose motor function is deteriorating and has received biological interface apparatus 100 prior to the motor impairment reaching a severe level. Patient 500 of FIG. 8 has received an FES device including FES stimulators 60, some of which are shown in a partial cutaway view of patient 500's right thigh muscles. FES stimulators are implanted in all muscles in which motor function is to be restored, such as in a majority of leg muscles for a paraplegic patient. Interface 135, shown attached near the patient's hip, includes a power supply, such as a rechargeable or replaceable battery, and may supply power to one or more components of apparatus 100. Interface 135 includes wireless transmission and receiving means, such as an RF transceiver, and can send and receive information to or from each FES stimulator, as well as processing unit second portion 130b. Interface 135 further includes multiple electronic components to perform mathematical computations or other signal processing functions, as well as provide memory storage. Interface 135 may provide a function of further processing the multicellular signals or a derivative of the multicellular signals.

The processed signals transmitted by processing unit second portion 130b are transmitted to the multiple FES stimulators 60, such as by way of interface 135, to cause muscle contractions such as those used to walk or change from a sitting to a standing position. In order for apparatus 100 to perform in a safe and reliable manner, one or more configuration routines, such as a calibration routine and a patient training routine stored in electronic memory of the processing unit, will be performed. The configuration routine may require the use of an operator, not the patient, such as physical therapist 110′ of FIG. 8. The patient training or other configuration routine, may involve configuration of the joint movement device, such as an exercise to determine patient range of motion. In a preferred embodiment, physical therapist 110′ records numerous parameters associated with acceptable patient movements, as well as angles, positions, forces and other factors to avoid. Physical therapist 110′ takes the information and manually enters this data such as by way of a configuration apparatus, as has been described in detail in reference to FIG. 5, which transmits the data to processing unit second portion 130b and/or interface 135.

In another preferred embodiment, apparatus 100 includes one or more integral physical therapy routines, such as routine that systematically increases a patient range. Information stored during each physical therapy event is captured either automatically, or manually as entered by physical therapist 110′. In another preferred embodiment, apparatus 100 includes one or more sensors, not shown, such as sensors whose signals are received by interface 135 and/or processing unit second portion 130b. An EMG sensor can be used to indicate a level of spasticity and/or a level of reflexivity used by apparatus 100 to improve a physical therapy event. A pressure sensor, force sensor or strain sensor may produce a signal that is compared to a threshold used to limit the processed signals to one or more minimums or maximums for values of controlled device performance.

Sensors may be used to monitor resistance to movement or amount of force required to perform a task. Physiologic sensors can be included such as a sensor selected from the group consisting of: EKG; respiration; blood glucose; temperature; blood pressure; EEG; perspiration; and combinations of the preceding. Output of the physiologic sensor can be used by the processing unit or a separate computational component of apparatus 100 to maintain the physical therapy within a range of values, avoid patient discomfort or potential adverse event. These systems may have one or more thresholds, such as adjustable thresholds, to detect irregular heart rate, nausea, pain, rise in blood pressure, and other adverse conditions. Physiologic data, as well as other recorded data can be stored and statistically trended between physical therapy events, again to optimize the therapy and/or avoid complications.

Referring now to FIG. 9, another preferred embodiment of a joint movement device of the present invention is illustrated, wherein a piston assembly has been fixedly attached to two bones of a patient to apply a torsional force to the joint attaching the two joints. FIG. 9 depicts a cutaway view of arm 510 of a patient, wherein joint movement device 90 has been implanted under the skin. Piston assembly 95 includes a proximal end, which is fixedly mounted to humerus bone 511 of arm 510 with bone screw 99. Piston assembly 95 includes a housing 98, which surrounds a lumen that exits the distal end of piston assembly 95, and slidingly receives a proximal end of piston 97. A linear actuator, such as a hydraulic or pneumatic assembly contained within housing 98, controllably advances and retracts piston 97. A majority of the length of piston 97 is contained within housing 98 at the fully retracted and fully advanced conditions. In a preferred embodiment, the maximum distance traveled by the piston is less than one inch. Other linear actuators include a rotational motor driven linear drive, and a shaped memory alloy in which a controllable contraction, such as via heating, is used in combination with a coil spring for advancement. The distal end of piston 97 is fixedly attached to two bones, radius 513 and ulna 512, with bone screws 99 such that advancement and retraction of piston 97 applies a torsional force to the elbow joint of arm 510. In an alternative embodiment, piston 97 or a portion of housing 98 may be inserted into a hollow or hollowed out portion of a bone, and secured by frictional engagement or an adhesive such as bone cement.

Joint movement device 90 further includes electronic module 96 which includes wireless data transfer means, computational and other signal processing functions, a power supply or a power receiving element such as an inductive coil, one or more sensors or sensor attachment means, and other functions appropriate for the secure control of joint movement device 90. A sensor may be incorporated into piston assembly 95 that is in communication with electronic module 96. Electronic module 96 preferably receives processed signals from the biological interface apparatus of the current invention, apparatus not shown, such that multicellular signals, such as cellular signals under voluntary control of the patient, are processed to produce processed signals to control joint movement device 90. In a preferred embodiment, at least a portion of the sensor of the biological interface apparatus is placed in a part of the patient's motor cortex that is associated with the limb being controlled by joint movement device 90.

Numerous methods are provided in the multiple embodiments of the disclosed invention. A preferred method embodiment includes a method of selecting a specific device to be controlled by the processed signals of a biological interface apparatus. The method comprises the steps of: providing a biological interface apparatus for collecting multicellular signals emanating from one or more living cells of a patient and for transmitting processed signals to control a device. The biological interface apparatus comprises: a sensor for detecting the multicellular signals, the sensor comprising a plurality of electrodes to allow for detection of the multicellular signals; a processing unit for receiving the multicellular signals from the sensor, for processing the multicellular signals to produce processed signals, and for transmitting the processed signals; a first controlled device for receiving the processed signals; a second controlled device for receiving the processed signals; and a selector module that is used to select the specific device to be controlled by the processed signals.

It should be understood that numerous other configurations of the systems, devices and methods described herein could be employed without departing from the spirit or scope of this application. It should be understood that the system includes multiple functional components, such as a sensor for detecting multicellular signals, a processing unit for processing the multicellular signals to produce processed signals, and the controlled device that is controlled by the processed signals. Different from the logical components are physical or discrete components, which may include a portion of a logical component, an entire logical component and combinations of portions of logical components and entire logical components. These discrete components may communicate or transfer data to or from each other, or communicate with devices outside the system. In each system, physical wires, such as electrical wires or optical fibers, can be used to transfer data between discrete components, or wireless communication means can be utilized. Each physical cable can be permanently attached to a discrete component, or can include attachment means to allow attachment and potentially allow, but not necessarily permit, detachment. Physical cables can be permanently attached at one end, and include attachment means at the other.

The sensors of the systems of this application can take various forms, including multiple discrete component forms, such as multiple penetrating arrays that can be placed at different locations within the body of a patient. The processing unit of the systems of this application can also be contained in a single discrete component or multiple discrete components, such as a system with one portion of the processing unit implanted in the patient, and a separate portion of the processing unit external to the body of the patient. The sensors and other system components may be utilized for short term applications, such as applications less than twenty four hours, sub-chronic applications such as applications less than thirty days, and chronic applications. Processing units may include various signal conditioning elements such as amplifiers, filters, signal multiplexing circuitry, signal transformation circuitry and numerous other signal processing elements. In a preferred embodiment, an integrated spike sorting function is included. The processing units performs various signal processing functions including but not limited to: amplification, filtering, sorting, conditioning, translating, interpreting, encoding, decoding, combining, extracting, sampling, multiplexing, analog to digital converting, digital to analog converting, mathematically transforming and/or otherwise processing cellular signals to generate a control signal for transmission to a controllable device. The processing unit utilizes numerous algorithms, mathematical methods and software techniques to create the desired control signal. The processing unit may utilize neural net software routines to map cellular signals into desired device control signals. Individual cellular signals may be assigned to a specific use in the system. The specific use may be determined by having the patient attempt an imagined movement or other imagined state. For most applications, it is preferred that that the cellular signals be under the voluntary control of the patient. The processing unit may mathematically combine various cellular signals to create processed signals for device control.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims. In addition, where this application has listed the steps of a method or procedure in a specific order, it may be possible, or even expedient in certain circumstances, to change the order in which some steps are performed, and it is intended that the particular steps of the method or procedure claim set forth herebelow not be construed as being order-specific unless such order specificity is expressly stated in the claim.

What is claimed is:

1. A joint movement device for applying a force to a patient's joint, said device comprising:
a force translating structure configured to be in contact with a portion of the patient;
at least one control cable with a proximal end and a distal end, the distal end attached to a portion of the force translating structure; and
a force producing assembly that is operably attached to the proximal end of the control cable;
wherein a force applied by said force producing assembly to the proximal end of said control cable is capable of causing a resultant force to be applied to the patient's joint.

2. The device of claim 1, wherein the patient's joint is a knee.

3. The device of claim 1, wherein the patient's joint is one of the patient's ankles.

4. The device of claim 3, wherein said device is capable of further applying a force to one or more of the patient's toe joints.

5. The device of claim 1, wherein the patient's joint is one of the patient's toe joints.

6. The device of claim 1, comprising a first control cable and a second control cable, wherein the first control cable and the second control cable are configured to be placed across a first joint of the patient.

7. The device of claim 6, wherein a force applied to the first control cable causes the first joint to flex in a first direction and a force applied to the second control cable causes the first joint to flex in a second direction, wherein the first direction and the second direction are substantially different.

8. The device of claim 7, wherein the first joint is selected from the group consisting of: ankle, shoulder, wrist, finger, and hip.

9. The device of claim 7, wherein the first direction is substantially opposite the second direction.

10. The device of claim 1, wherein said device is configured to grasp an object.

11. The device of claim 1, wherein the resultant force is applied to a wrist of the patient.

12. The device of claim 1, wherein the resultant force is applied to a finger joint of the patient.

13. The device of claim 1, wherein the portion of the patient includes one of the patient's hands or wrists.

14. The device of claim 1, wherein the portion of the patient includes one of the patient's fingers.

15. The device of claim 14, wherein the portion of the patient includes multiple fingers of the patient.

16. The device of claim 1, wherein the force translating structure includes an elastic portion.

17. The device of claim 1, wherein the force translating structure includes a flexible portion.

18. The device of claim 1, wherein the force translating structure has a glove configuration.

19. The device of claim 1, wherein the force translating structure has a sock configuration.

20. The device of claim 1, wherein the force translating structure is configured to substantially surround one of the forearms of the patient.

21. The device of claim 1, wherein the force translating structure is configured to substantially surround one of the wrists of the patient.

22. The device of claim 1, wherein the force translating structure is configured to substantially surround at least one finger of the patient.

23. The device of claim 1, wherein the force translating structure includes a power supply.

24. The device of claim 1, wherein the force translating structure includes one or more longitudinal coverings, each covering fixedly attached to said force translating structure such that a passageway is formed between the force translating structure and the longitudinal covering, said passageway slidingly receiving at least one control cable.

25. The device of claim 24, wherein at least one passageway is short relative to the length of the control cable.

26. The device of claim 25, wherein multiple passageways surround a single control cable.

27. The device of claim 24, wherein at least one passageway is more than half the length of the control cable slidingly received within said passageway.

28. The device of claim 27, wherein said control cable is slidingly received in a single passageway.

29. The device of claim 24, comprising a second control cable, said second control cable slidingly received in the passageway that slidingly receives the first control cable.

30. The device of claim 24, comprising a second control cable and a second longitudinal covering, said second control cable being slidingly received by a passageway formed by the second longitudinal covering.

31. The device of claim 24, wherein at least one longitudinal covering causes at least one control cable to remain in close proximity to the force translating structure from a location proximate the force producing assembly to a location proximate the end of a finger of the patient when a force is applied to the proximal end of the control cable by the force producing assembly.

32. The device of claim 31, wherein said applied force is capable of causing a finger to curl inward.

33. The device of claim 24, wherein at least one longitudinal covering causes at least one control cable to remain in close proximity to the force translating structure from a location proximate the force producing assembly to a location proximate the first joint of a finger of the patient when a force is applied to the proximal end of the control cable by the force producing assembly.

34. The device of claim 33, wherein said applied force is capable of causing a wrist to curl inward.

35. The device of claim 34, wherein the finger is selected from the group consisting of: middle finger; index finger; and fourth finger.

36. The device of claim 24, further comprising a spring member, and the force translating structure has a top surface and a bottom surface, wherein at least one longitudinal covering is fixedly attached to a portion of the bottom surface, and said spring member is fixedly attached to one or more portions of the top surface.

37. The device of claim 36, wherein the spring member is resiliently biased in a straight or curved configuration.

38. The device of claim 37, wherein the spring member is fixedly attached to a portion of the force translating structure which is configured to be in proximity to one or more joints of the patient such that said joints are resiliently biased relative to said spring member.

39. The device of claim 38, wherein the one or more joints are resiliently biased by the spring member in a first direction, and the force applied by the force producing assembly to the control cable causes at least one or the joints to move in a relatively opposite direction.

40. The device of claim 38, wherein the joints include finger joints, and at least one finger joint is resiliently biased to be in a curved configuration.

41. The device of claim 38, wherein the joints include finger joints, and at least one finger joint is resiliently biased to be in a relatively straight configuration.

42. The device of claim 1, further comprising a constraining band, said constraining band circumferentially positioned in proximity to a joint of the patient.

43. The device of claim 42, wherein the force translating structure is more elastic than the constraining band.

44. The device of claim 42, wherein the constraining band is constructed of materials to exhibit minimal stretch.

45. The device of claim 42, wherein the constraining band causes the control cable to flex at a joint location when force is applied to the proximal end of said control cable.

46. The device of claim 42, wherein the constraining band is configured to be located at one or more finger joints.

47. The device of claim 42, wherein the constraining band is configured to be located at a wrist of the patient.

48. The device of claim 1, wherein the control cable is constructed of materials to avoid stretching.

49. The device of claim 48, wherein the control cable is constructed of a flurorocarbon.

50. The device of claim 1, wherein the control cable is a monofilament material.

51. The device of claim 1, wherein the control cable is constructed of Nitinol wire.

52. The device of claim 1, further comprising a second control cable.

53. The device of claim 52, wherein the at least one control cable is used to move a finger joint, and the second control cable is used to move a wrist joint of the same hand of the patient as the finger joint.

54. The device of claim 1, wherein the force producing assembly includes a rotational motor.

55. The device of claim 54, wherein the rotational motor is selected from the group consisting of: a stepper motor; a DC motor; an AC motor; a synchronous motor; and combinations thereof.

56. The device of claim 55, wherein the rotational motor is a stepper motor, said stepper motor including a holding detent force.

57. The device of claim 54, wherein the rotational motor includes a position encoder.

58. The device of claim 57, wherein the position encoder is an optical encoder.

59. The device of claim 54, wherein the motor is operably attached to and causes the rotation of an axle, said axle including one or more pulleys along its length.

60. The device of claim 59, wherein the axle includes a first pulley and a second pulley, said first pulley having a larger diameter than the second pulley.

61. The device of claim 60, wherein the first pulley is operably attached to a first control cable which when force is applied to said first control cable the resultant force causes a middle finger joint to rotate, and wherein the second pulley is operably attached to a second control cable which when force is applied to said second control cable the resultant force causes a little finger joint to rotate.

62. The device of claim 60, wherein the first pulley is operably attached to a first control cable which when force is applied to said first control cable the resultant force causes a first joint to rotate through a first angle, and wherein the second pulley is operably attached to a second control cable which when force is applied to said second control cable the resultant force causes a second joint to rotate through a second angle, wherein said first angle is greater than said second angle.

63. The device of claim 59, wherein at least one pulley is releasable attached to the axle.

64. The device of claim 63, wherein the at least one pulley is attached to the pulley by activation of a clutch assembly.

65. The device of claim 63, wherein the at least one pulley is normally unattached to the axle.

66. The device of claim 1, wherein the force producing assembly includes a linear actuator.

67. The device of claim 66, wherein the linear actuator is selected from the group consisting of: a solenoid; a Nitinol wire; and combinations thereof.

68. The device of claim 1, wherein the force producing assembly includes a mechanical advantage assembly.

69. The device of claim 68, wherein the mechanical advantage assembly includes a component selected from the group consisting of: a lever arm; a cam; a pneumatic assembly; a hydraulic assembly; and combinations thereof.

70. The device of claim 1, wherein the resultant force is a torsional force.

71. The device of claim 1, wherein the resultant force is a linear force.

72. The device of claim 1, wherein the resultant force does not substantially change the angular position of the joint to which the resultant force is applied.

73. The device of claim 72, wherein the resultant force causes an object to be grasped by the patient.

74. The device of claim 1, wherein the resultant force causes angular displacement of the joint to which the resultant force is applied.

75. The device of claim 1, wherein the resultant force places the joint to which the resultant force is applied in tension.

76. The device of claim 75, wherein the joint is a joint of the patient's hand, and the resultant force causes a gripping force of said hand.

77. The device of claim 1, wherein the resultant force causes a wrist of the patient to curl inward.

78. The device of claim 1, wherein the resultant force causes a finger of the patient to curl inward.

79. The device of claim 1, further comprising a power supply.

80. The device of claim 79, wherein the power supply supplies power to the force producing assembly.

81. The device of claim 1, further comprising a sensor.

82. The device of claim 81, wherein the sensor provides a signal related to the resultant force.

83. The device of claim 82, wherein the sensor provides a signal related to the tension in one or more control cables.

84. The device of claim 81, wherein a signal provided by the sensor is compared to a threshold value that prevents the resultant force from exceeding a pre-determined level.

85. The device of claim 84, wherein the threshold value is adjustable by an operator of the system.

86. The device of claim 85, wherein the operator is the patient.

87. A joint movement device for applying force to a patient's elbow and at least one joint of the patient's hand, said device comprising:
a torque generating assembly configured for applying a torsional force to the patient's elbow; and
a force generator configured for applying a force to the at least one joint of the patient's hand including a patient's wrist and/or finger joint, the force generator comprising:
a force translating structure configured to be attached to a portion of the patient;
at least one control cable with a proximal end and a distal end, the distal end attached to a portion of said force translating structure; and
a force producing assembly that is operably attached to the proximal end of the control cable;
wherein a force applied by said force producing assembly to the proximal end of said control cable causes a resultant force to be applied to the at least one joint of the patient's hand.

88. The device of claim 87, wherein the torque generating assembly includes a rotational motor.

89. The device of claim 88, wherein the rotational motor is selected from the group consisting of: a stepper motor; a DC motor; an AC motor; a synchronous motor; and combinations thereof.

90. The device of claim 89, wherein the rotational motor is a stepper motor, said stepper motor including a holding detent force.

91. The device of claim 88, wherein the rotational motor includes a position encoder.

92. The device of claim 91, wherein the position encoder is an optical encoder.

93. The device of claim 87, wherein the torque generating assembly includes a power supply.

94. The device of claim 93, wherein the power supply is a rechargeable battery.

95. The device of claim 87, wherein the torque generating assembly includes a mechanical advantage assembly.

96. The device of claim 95, wherein the mechanical advantage assembly includes a mechanical advantage selected from the group consisting of: an assembly of gears; a cam assembly; a lever arm assembly; and combinations thereof.

97. The device of claim 87, wherein said device is configured to grasp an object.

98. The device of claim 87, wherein the resultant force is configured to be applied to a wrist of the patient.

99. The device of claim 87, wherein the resultant force is configured to be applied to a finger joint of the patient.

100. The device of claim 87, wherein the portion of the patient includes one of the patient's hands or wrists.

101. The device of claim 87, wherein the portion of the patient includes one of the patient's fingers.

102. The device of claim 101, wherein the portion of the patient includes multiple fingers of the patient.

103. The device of claim 87, wherein the force translating structure includes an elastic portion.

104. The device of claim 87, wherein the force translating structure includes a flexible portion.

105. The device of claim 87, wherein the force translating structure has a glove configuration.

106. The device of claim 87, wherein the force translating structure has a sock configuration.

107. The device of claim 87, wherein the force translating structure is configured to substantially surround one of the forearms of the patient.

108. The device of claim 87, wherein the force translating structure is configured to substantially surround one of the wrists of the patient.

109. The device of claim 87, wherein the force translating structure is configured to substantially surround at least one finger of the patient.

110. The device of claim 87, wherein the force translating structure includes a power supply.

111. The device of claim 87, wherein the force translating structure includes one or more longitudinal coverings, each covering fixedly attached to said force translating structure such that a passageway is formed between the force translating structure and the longitudinal covering, said passageway slidingly receiving at least one control cable.

112. The device of claim 111, wherein at least one passageway is short relative to the length of the control cable.

113. The device of claim 112, wherein multiple passageways surround a single control cable.

114. The device of claim 111, wherein at least one passageway is more than half the length of the control cable slidingly received within said passageway.

115. The device of claim 114, wherein said control cable is slidingly received in a single passageway.

116. The device of claim 111, comprising a second control cable, said second control cable slidingly received in the passageway that slidingly receives the first control cable.

117. The device of claim 111, comprising a second control cable and a second longitudinal covering, said second control cable being slidingly received by a passageway formed by the second longitudinal covering.

118. The device of claim 111, wherein at least one longitudinal covering causes at least one control cable to remain in close proximity to the force translating structure from a location proximate the force producing assembly to a location proximate the end of a finger of the patient when a force is applied to the proximal end of the control cable by the force producing assembly.

119. The device of claim 118, wherein said applied force is capable of causing a finger to curl inward.

120. The device of claim 111, wherein at least one longitudinal covering causes at least one control cable to remain in close proximity to the force translating structure from a location proximate the force producing assembly to a location proximate the first joint of a finger of the patient when a force is applied to the proximal end of the control cable by the force producing assembly.

121. The device of claim 120, wherein said applied force is capable of causing a wrist to curl inward.

122. The device of claim 121, wherein the finger is selected from the group consisting of: middle finger; index finger; and fourth finger.

123. The device of claim 111, further comprising a spring member, and the force translating structure has a top surface and a bottom surface, wherein at least one longitudinal covering is fixedly attached to a portion of the bottom surface, and said spring member is fixedly attached to one or more portions of the top surface.

124. The device of claim 123, wherein the spring member is resiliently biased in a straight or curved configuration.

125. The device of claim 124, wherein the spring member is fixedly attached to a portion of the force translating structure which is configured to be in proximity to one or more joints of the patient such that said joints are resiliently biased relative to said spring member.

126. The device of claim 125, wherein the one or more joints are resiliently biased by the spring member in a first direction, and the force applied by the force producing assembly to the control cable causes at least one or the joints to move in a relatively opposite direction.

127. The device of claim 125, wherein the joints include finger joints, and at least one finger joint is resiliently biased to be in a curved configuration.

128. The device of claim 125, wherein the joints include finger joints, and at least one finger joint is resiliently biased to be in a relatively straight configuration.

129. The device of claim 87, further comprising a constraining band, said constraining band circumferentially positioned in proximity to a joint of the patient.

130. The device of claim 129, wherein the force translating structure is more elastic than the constraining band.

131. The device of claim 129, wherein the constraining band is constructed of materials to exhibit minimal stretch.

132. The device of claim 129, wherein the constraining band causes the control cable to flex at a joint location when force is applied to the proximal end of said control cable.

133. The device of claim 129, wherein the constraining band is configured to be located at one or more finger joints.

134. The device of claim 129, wherein the constraining band is configured to be located at a wrist of the patient.

135. The device of claim 87, wherein the control cable is constructed of materials to avoid stretching.

136. The device of claim 135, wherein the control cable is constructed of a flurorocarbon.

137. The device of claim 87, wherein the control cable is a monofilament material.

138. The device of claim 87, wherein the control cable is constructed of Nitinol wire.

139. The device of claim 87, further comprising a second control cable.

140. The device of claim 139, wherein the at least one control cable is used to move a finger joint, and the second control cable is used to move a wrist joint of the same hand of the patient as the finger joint.

141. The device of claim 87, wherein the force producing assembly includes a rotational motor.

142. The device of claim 141, wherein the rotational motor is selected from the group consisting of: a stepper motor; a DC motor; an AC motor; a synchronous motor; and combinations thereof.

143. The device of claim 142, wherein the rotational motor is a stepper motor, said stepper motor including a holding detent force.

144. The device of claim 141, wherein the rotational motor includes a position encoder.

145. The device of claim 144, wherein the position encoder is an optical encoder.

146. The device of claim 141, wherein the motor is operably attached to and causes the rotation of an axle, said axle including one or more pulleys along its length.

147. The device of claim 146, wherein the axle includes a first pulley and a second pulley, said first pulley having a larger diameter than the second pulley.

148. The device of claim 147, wherein the first pulley is operably attached to a first control cable which when force is applied to said first control cable the resultant force causes a middle finger joint to rotate, and wherein the second pulley is operably attached to a second control cable which when force is applied to said second control cable the resultant force causes a little finger joint to rotate.

149. The device of claim 147, wherein the first pulley is operably attached to a first control cable which when force is applied to said first control cable the resultant force causes a first joint to rotate through a first angle, and wherein the second pulley is operably attached to a second control cable which when force is applied to said second control cable the resultant force causes a second joint to rotate through a second angle, wherein said first angle is greater than said second angle.

150. The device of claim 146, wherein at least one pulley is releasable attached to the axle.

151. The device of claim 150, wherein the at least one pulley is attached to the pulley by activation of a clutch assembly.

152. The device of claim 150, wherein the at least one pulley is normally unattached to the axle.

153. The device of claim 87, wherein the force producing assembly includes a linear actuator.

154. The device of claim 153, wherein the linear actuator is selected from the group consisting of: a solenoid; a Nitinol wire; and combinations thereof.

155. The device of claim 87, wherein the force producing assembly includes a mechanical advantage assembly.

156. The device of claim 155, wherein the mechanical advantage assembly includes a component selected from the group consisting of: a lever arm; a cam; a pneumatic assembly; a hydraulic assembly; and combinations thereof.

157. The device of claim 87, wherein the resultant force is a torsional force.

158. The device of claim 87, wherein the resultant force is a linear force.

159. The device of claim 87, wherein the resultant force does not substantially change the angular position of the joint to which the resultant force is applied.

160. The device of claim 159, wherein the resultant force causes an object to be grasped by the patient.

161. The device of claim 87, wherein the resultant force causes angular displacement of the joint to which the resultant force is applied.

162. The device of claim 87, wherein the resultant force places the joint to which the resultant force is applied in tension.

163. The device of claim 162, wherein the joint is a joint of the patient's hand, and the resultant force causes a gripping force of said hand.

164. The device of claim 87, wherein the resultant force causes a wrist of the patient to curl inward.

165. The device of claim 87, wherein the resultant force causes a finger of the patient to curl inward.

166. The device of claim 87, further comprising a power supply.

167. The device of claim 166, wherein the power supply supplies power to the force producing assembly.

168. The device of claim 87, further comprising a sensor.

169. The device of claim 168, wherein the sensor provides a signal related to the resultant force.

170. The device of claim 169, wherein the sensor provides a signal related to the tension in one or more control cables.

171. The device of claim 168, wherein a signal provided by the sensor is compared to a threshold value that prevents the resultant force from exceeding a pre-determined level.

172. The device of claim 171, wherein the threshold value is adjustable by an operator of the system.

173. The device of claim 172, wherein the operator is the patient.

174. A movement assist system for applying a force to one or more joints of a patient, said system comprising;
the joint movement device of claim 1; and
a biological interface apparatus comprising: a sensor comprising a plurality of electrodes for detecting multicellular signals; and a processing unit configured to receive the multicellular signals from the sensor, process the multicellular signals to produce a processed signal, and transmit the processed signal to the joint movement device.

175. The device of claim 174, wherein the multicellular signals emanate from nerve cells associated with movement of the one or more joints receiving the force from the movement device.

176. The device of claim 175, wherein the sensor is not placed in the brain of the patient.

177. The device of claim 175, wherein the sensor is configured to be placed in the spinal cord of the patient.

178. The device of claim 174, wherein the multicellular signals emanate from neurons of the motor cortex of the patient.

179. The device of claim 178, wherein the neurons are associated with the hand area of the patient cortex.

180. The device of claim 179, wherein the joints receiving the force from the movement device are part of the hand corresponding to that area of the motor cortex.

181. The device of claim 178, wherein the neurons are associated with the foot area of the patient cortex.

182. The device of claim 181, wherein the joints receiving the force from the movement device are part of the foot corresponding to that area of the motor cortex.

183. The device of claim 174, wherein the system allows patient voluntary movement of a limb of said patient.

184. The device of claim 174, wherein the system allows patient voluntary movement of a digit of said patient.

185. The device of claim 174, wherein the system allows patient voluntary causation of grip force of a hand of said patient.

186. The device of claim 174, wherein the said system allows patient voluntary movement of multiple joints of the patient.

187. The device of claim 174, further comprising a feedback module for providing movement device data to said system.

188. The device of claim 187, wherein the movement device data is provided to the biological interface apparatus.

189. The device of claim 187, wherein the movement device further comprises an additional sensor, the movement device data comprising the signal provided by said additional sensor.

190. The device of claim 174, wherein said system is a neural interface system.

191. The device of claim 174, wherein said system is a brain machine interface.

192. The device of claim 174, wherein said system is configured to change states due to a change in state of a monitored biological signal of the patient.

193. The device of claim 192, wherein the change in system state is selected from the group consisting of: system on or off state; calibration routine on or off state; reset routine on or off state; and combinations thereof.

194. The device of claim 192, wherein the monitored biological signal is selected from the group consisting of: eye motion; eyelid motion; facial muscle activation or other electromyographic activity; heart rate; EEG; LFP; respiration; and combinations thereof.

195. The device of claim 192, wherein the monitored biological signal is a time code of brain activity.

196. The device of claim 174, further comprising a patient activated input device, wherein said system is configured to change state due to a signal received from said patient activated input device.

197. The device of claim 196, wherein the patient activated input device is selected from the group consisting of: chin joystick; Eyebrow EMG switch; EEG activated switch; eye tracker; head tracker; neck movement switch; shoulder movement switch; sip-and-puff joystick controller; speech recognition switch; tongue switch such as a tongue palate switch; and combinations thereof.

198. The device of claim 174, wherein the multicellular signals emanate from the central nervous system of the patient.

199. The device of claim 174, wherein the multicellular signals consist of one or more of: neuron spikes; ECoG signals; LFP signals; and EEG signals.

200. The device of claim 174, wherein the patient is a human being.

201. The device of claim 174, wherein the patient is selected from the group consisting of: a quadriplegic; a paraplegic; an amputee; a spinal cord injury victim; a physically impaired person; an ALS patient; and combinations thereof.

202. The device of claim 174, wherein the patient is healthy and or otherwise is not utilizing said system to provide a therapeutic or restorative function.

203. The device of claim 202, wherein the patient is utilizing the system to increase hand strength.

204. The device of claim 174, wherein the sensor includes at least one multi-electrode array, said multi-electrode array including a plurality of electrodes.

205. The device of claim 204, wherein the plurality of electrodes are configured to penetrate into neural tissue of the brain to detect electric signals generated from neurons.

206. The device of claim 204, wherein the multi-electrode array includes at least one of: a recording electrode; a stimulating electrode; and an electrode having recording and stimulating capabilities.

207. The device of claim 204, wherein the sensor further comprises a second multi-electrode array.

208. The device of claim 174, wherein the sensor includes multiple wires or wire bundle electrodes.

209. The device of claim 174, wherein the sensor includes electrodes incorporated into one or more of: a subdural grid; a scalp electrode; a wire electrode; and a cuff electrode.

210. The device of claim 174, wherein the sensor includes two or more discrete components.

211. The device of claim 210, wherein each of said discrete components includes one or more electrodes.

212. The device of claim 210, wherein each of the discrete components is comprised of one or more of the following: a multi-electrode array; a wire or wire bundle; a subdural grid; and a scalp electrode.

213. The device of claim 174, wherein the plurality of electrodes are capable of recording from clusters of neurons and outputting detected signals comprising multiple neuron signals.

214. The device of claim 213, wherein detected signals are a measure of the LFP response from neural activity.

215. The device of claim 213, wherein the multiple neuron signals comprise one or more of: ECoG signals; LFP signals; EEG signals; and peripheral nerve signals.

216. The device of claim 174, wherein one or more electrodes are placed into tissue selected from the group consisting of: nerve tissue; organ tissue; tumor tissue; other tissue; and combinations thereof.

217. The device of claim 174, wherein the processing unit performs one or more of: amplifying; filtering; sorting; conditioning; computing; translating; interpreting; encoding; decoding; combining; extracting; sampling; multiplexing; analog to digital converting; digital to analog converting; mathematically transforming and/or otherwise processing cellular signals to generate a control signal for transmission to a controlled device.

218. The device of claim 174, wherein the processing unit includes one or more of: a microprocessor or microcontroller; a temperature sensor; a pressure sensor; a strain gauge; an accelerometer; a volume sensor; an electrode; an array of electrodes; an audio transducer; a mechanical vibrator; a drug delivery device; a magnetic field generator; a photo detector element; a camera or other visualization apparatus; a wireless communication element; a light producing element; an electrical stimulator; a physiologic sensor; a heating element; and a cooling element.

219. The device of claim 174, further comprising a controlled device.

220. The device of claim 174, further comprising a stimulating device.

221. The device of claim 174, further comprising a patient feedback module.

222. The device of claim 221, wherein the patient feedback module includes one or more of: an audio transducer, a tactile transducer, a visual transducer, a video display, a gustatory transducer; and an olfactory transducer.

223. The device of claim 221, wherein the patient feedback module includes a stimulator, and one or more neurons are stimulated to cause movement or sensation in a part of the patient's body.

224. The device of claim 174, further comprising a drug delivery system, wherein the processing unit sends a signal to the drug delivery system to deliver a therapeutic agent or drug to at least a portion of the patient's body.

225. The device of claim 174, further comprising an embedded ID.

226. The device of claim 225, wherein the embedded ID is used to confirm compatibility of one or more discrete components of the system.
